(19) United States
(12) Patent Application Publication
(10) Pub. No.: US 2021/0047428 A1
(43) Pub. Date: Feb. 18, 2021

(54) METHOD FOR GENERATING ANTIBODIES WITH IMPROVED SPECIFICITY AND/OR AFFINITY

(71) Applicant: Garvan Institute of Medical Research, New South Wales (AU)

(72) Inventors: Daniel CHRIST, New South Wales (AU); Robert BRINK, New South Wales (AU); Christopher GOODNOW, New South Wales (AU); Deborah BURNETT, New South Wales (AU); Peter SCHOFIELD, New South Wales (AU)

(73) Assignee: Garvan Institute of Medical Research, New South Wales (AU)

(21) Appl. No.: 17/043,044

(22) PCT Filed: Mar. 29, 2019
(86) PCT No.: PCT/AU2019/050283

§ 371 Date: Sep. 29, 2020

(30) Foreign Application Priority Data
Mar. 29, 2018 (AU) 2018901070

(51) Int. Cl. C07K 16/30; A01K 67/027; C12N 5/0781

(52) U.S. Cl. CPC C07K 16/30; A01K 67/0275; C12N 5/0635; A01K 2207/12; C07K 2317/92; A01K 2227/105; A01K 2267/01; C12N 2510/02; A01K 2217/05

(57) ABSTRACT

The present disclosure relates to methods of generating antibodies with improved specificity and/or affinity for a target antigen, as well as B-cells and hybridomas expressing the antibodies and compositions comprising antibodies with improved specificity and/or affinity for the target antigen.

### CROSS-REFERENCE TO RELATED APPLICATIONS

The present application claims priority from Australian Provisional Patent Application No 2018901070 filed on 29 Mar. 2018, the content of which is incorporated herein by reference.

### TECHNICAL FIELD

The present disclosure relates to methods of generating antibodies with improved specificity and/or affinity for a target antigen, as well as B-cells and hybridomas expressing the antibodies and compositions comprising antibodies with improved specificity and/or affinity for the target antigen.

### BACKGROUND

In some instances, antibodies can distinguish nearly identical foreign and self-antigens, such as the glycolipids on the cell wall of Campylobacter jejuni and those on human nerve cells, with less than 0.1% of infected people producing cross-reactive antibodies that result in paralysis and Guillain-Barré Syndrome. However, apparent limits to antibody self-foreign discrimination are exploited by Human Immunodeficiency Virus (HIV), Lymphocytic choriomeningitis virus and Lassa fever viruses. They establish persistent infections and evade antibodies by mimicking self and cloaking their foreign envelope proteins with self-glycans.

Furthermore, functionally important vaccine targets are often highly disordered, or surrounded by flexible loops making effective affinity maturation to complex antigens or conformationally-flexible antigens even more challenging.

Immunization of mice or rats with a “non-self” protein is a commonly used method to obtain monoclonal antibodies, and relies on the immune system's ability to recognize the immunogen as foreign. Tolerance, the ability of the immune system to prevent responses to self-antigens, makes it difficult to generate a strong immune response in mice with a mouse self-antigen or highly conserved human antigen. Specific knockout mice are often used to overcome the immune tolerance associated with self-antigens.

Accordingly, there remains a need for methods of generating antibodies to self-antigens, highly conserved human antigens and/or conformationally-flexible antigens, and/or methods of generating antibodies with improved specificity and/or affinity for a target antigen.

### SUMMARY

The method described herein can be used to produce antibodies with desirable specificity and affinity to a target antigen in an animal by introducing the target antigen into an animal expressing a structurally related antigen.

Accordingly, in one aspect, there is provided a method for generating an antibody having improved specificity and/or affinity for a target antigen, the method comprising:
i) introducing the target antigen in an animal, wherein the animal expresses a variant antigen that is structurally related to the target antigen, and the animal further comprises B-cells encoding an antibody with a basic specificity and/or affinity for the target antigen, whereby the B-cells express a B-cell receptor comprising the antibody;
ii) allowing the B-cells to undergo affinity maturation in the animal; and
iii) isolating B-cells that have undergone affinity maturation in the animal;

whereby the B-cells isolated in iii) express antibodies with improved specificity and/or affinity for the target antigen compared to antibodies expressed by the B-cells prior to affinity maturation.

Accordingly, in another aspect there is provided a method for generating an antibody having improved specificity and/or affinity for a conformationally-flexible target antigen, the method comprising:
i) introducing the target antigen in an animal, wherein the animal expresses a variant antigen that is structurally related to the target antigen, and the animal further comprises B-cells encoding an antibody with a basic specificity and/or affinity for the target antigen, whereby the B-cells express a B-cell receptor comprising the antibody;
ii) allowing the B-cells to undergo affinity maturation in the animal; and
iii) isolating B-cells that have undergone affinity maturation in the animal;

whereby the B-cells isolated in iii) express antibodies with improved specificity and/or affinity for the target antigen compared to antibodies expressed by the B-cells prior to affinity maturation.

Accordingly, in another aspect there is provided a method for generating an antibody having improved specificity and/or affinity for a target antigen, the method comprising:
i) introducing into an animal a B-cell, the B-cell encoding an antibody with a basic specificity and/or affinity for the target antigen, whereby the B-cell expresses a B-cell receptor comprising the antibody, and wherein the animal expresses a variant antigen that is structurally related to the target antigen;
ii) introducing the target antigen in the animal;
iii) allowing the B-cells to undergo affinity maturation in the animal; and
iv) isolating the B-cells that have undergone affinity maturation in the animal;

whereby the B-cells isolated in iv) express antibodies with improved specificity and/or affinity for the target antigen compared to antibodies expressed by the B-cells prior to affinity maturation.

Accordingly in another aspect, there is provided a method for generating an antibody having improved specificity and/or affinity for a conformationally-flexible target antigen, the method comprising:
i) introducing into an animal a B-cell, the B-cell encoding an antibody with a basic specificity and/or affinity for the target antigen, whereby the B-cell expresses a B-cell receptor comprising the antibody, and wherein the animal expresses a variant antigen that is structurally related to the target antigen;
ii) introducing the target antigen in the animal;
iii) allowing the B-cells to undergo affinity maturation in the animal; and
iv) isolating the B-cells that have undergone affinity maturation in the animal;

whereby the B-cells isolated in iv) express antibodies with improved specificity and/or affinity for the target antigen compared to antibodies expressed by the B-cells prior to affinity maturation.

In one embodiment, the B-cells have been genetically modified to encode the antibody with the basic specificity and/or affinity for the target antigen.

In one embodiment, introducing the B-cell into the animal comprises irradiating the animal and transplanting the B-cells into the animal.

In one embodiment, the basic affinity of the antibody for the target antigen is lower affinity relative to a desired affinity.

In yet another embodiment, the lower affinity for the target antigen is a K_{D} of about 10⁻⁶ M to about 10⁻⁸ M.

In another embodiment, the improved affinity is an increase in affinity for the target antigen of at least 100-fold.

In yet another embodiment, the improved affinity is an increase in affinity for the target antigen of at least 1000-fold.

In one particular embodiment, the improved specificity for the target antigen is an at least 10-fold increase in specificity.

In one embodiment, the improved specificity for the target antigen is an at least 100-fold increase in specificity.

In yet another embodiment, the improved specificity for the target antigen is an at least 1000-fold increase in specificity.

In one embodiment, the target antigen is a peptide or polypeptide antigen.

In one embodiment, the variant antigen comprises at least one variant amino acid compared to the target antigen.

In another embodiment, the variant antigen comprises at least one variant amino acid residue in an epitope to which the antibody binds.

In yet another embodiment, the variant antigen comprises at least one variant amino acid residue in a surface that contacts the antibody heavy chain.

In another embodiment, the variant antigen is encoded by a transgene in the animal.

In one particular embodiment, the transgene comprises a ubiquitin promoter for expression of the variant antigen.

In yet another embodiment, the target antigen is a carbohydrate antigen or hapten.

In one embodiment, the carbohydrate antigen is selected from a glycoprotein, glycolipid, polysaccharide and glycoconjugate antigen.

In another embodiment, the carbohydrate antigen is a cancer cell antigen.

In one particular embodiment, the target antigen is a cancer neo-antigen.

In another embodiment, the carbohydrate antigen is a bacterial or viral carbohydrate antigen.

In one embodiment of the method described herein, the animal is a mouse or rat.

In one aspect, there is provided a method for generating an antibody having improved specificity and/or affinity for a target antigen, the method comprising:
i) genetically modifying a B-cell to encode an antibody with a basic specificity and/or affinity for the target antigen, whereby the B-cell expresses a B-cell receptor comprising the antibody;
ii) introducing the genetically modified B-cell in i) into an animal expressing or inoculated with the target antigen and expressing a variant antigen that is structurally related to the target antigen;
iii) allowing the B-cells to undergo affinity maturation in the animal; and
iv) isolating B-cells that have undergone affinity maturation in the animal;
whereby the B-cells isolated in iv) express antibodies with improved specificity and/or affinity for the target antigen compared to antibodies expressed by the B-cells prior to affinity maturation.

In another aspect, there is provided a method for generating an antibody having improved specificity and/or affinity for a conformationally-flexible target antigen, the method comprising:
i) genetically modifying a B-cell in an animal to encode an antibody with a basic specificity and/or affinity for the target antigen, whereby the B-cell expresses a B-cell receptor comprising the antibody, and wherein the animal expresses or is inoculated with the target antigen and expresses a variant antigen that is structurally related to the target antigen,
ii) allowing the B-cells to undergo affinity maturation in the animal; and
iii) isolating B-cells that have undergone affinity maturation in the animal;
whereby the B-cells isolated in iii) express antibodies with improved specificity and/or affinity for the target antigen compared to antibodies expressed by the B-cells prior to affinity maturation.

In yet another aspect, there is provided a method for providing an antibody having improved specificity and/or affinity for a target antigen, the method comprising:
i) identifying an antibody having a basic affinity for the target antigen;
ii) genetically modifying a B-cell to encode the antibody identified in i);
iii) introducing the genetically modified B-cell in ii) to an animal expressing the target antigen and expressing a variant antigen that is structurally related to the target antigen;
iv) allowing the B-cells to undergo affinity maturation in the animal; and
v) isolating B-cells that have undergone affinity maturation in the animal;
whereby the B-cells isolated in v) express antibodies with improved specificity and/or affinity for the target antigen compared to the variant antigen when compared to antibodies expressed by the B-cells prior to affinity maturation.

In yet another aspect, there is provided a method for generating an antibody having improved specificity and/or affinity for a conformationally-flexible target antigen, the method comprising:
i) genetically modifying a B-cell to encode an antibody with a basic specificity and/or affinity for the target antigen, whereby the B-cell expresses a B-cell receptor comprising the antibody;
ii) introducing the genetically modified B-cell in i) into an animal expressing or inoculated with the target antigen and expressing a variant antigen that is structurally related to the target antigen;
iii) allowing the B-cells to undergo affinity maturation in the animal; and
iv) isolating B-cells that have undergone affinity maturation in the animal;
whereby the B-cells isolated in iv) express antibodies with improved specificity and/or affinity for the target antigen compared to antibodies expressed by the B-cells prior to affinity maturation.

In yet another aspect, there is provided a method for generating an antibody having improved specificity and/or affinity for a target antigen, the method comprising:
i) genetically modifying a B-cell in an animal to encode an antibody with a basic specificity and/or affinity for the target antigen, whereby the B-cell expresses a B-cell receptor comprising the antibody, and wherein the animal expresses or is inoculated with the target antigen and expresses a variant antigen that is structurally related to the target antigen,
ii) allowing the B-cells to undergo affinity maturation in the animal; and
iii) isolating B-cells that have undergone affinity maturation in the animal;
whereby the B-cells isolated in iii) express antibodies with improved specificity and/or affinity for the target antigen compared to antibodies expressed by the B-cells prior to affinity maturation.

In yet another aspect, there is provided a method for providing an antibody having improved specificity and/or affinity for a conformationally-flexible target antigen, the method comprising:
i) identifying an antibody having a basic affinity for the target antigen;
ii) genetically modifying a B-cell to encode the antibody identified in i);
iii) introducing the genetically modified B-cell in ii) to an animal expressing the target antigen and expressing a variant antigen that is structurally related to the target antigen;
iv) allowing the B-cells to undergo affinity maturation in the animal; and
v) isolating B-cells that have undergone affinity maturation in the animal;
whereby the B-cells isolated in v) express antibodies with improved specificity and/or affinity for the target antigen compared to the variant antigen when compared to antibodies expressed by the B-cells prior to affinity maturation.

In one embodiment, the target antigen is a conformationally-flexible antigen. In another embodiment, the conformationally-flexible antigen is selected from the group consisting of: HIV envelope protein, circumsporozoite protein (CSP), merozoite surface protein 2 (MSP2) and GAD65.

In another aspect, there is provided a B-cell isolated according to the method described herein.

In a further aspect, there is provided an isolated antibody expressed by the isolated B-cell as described herein.

In yet another aspect, there is provided a hybridoma that is derived from the isolated B-cell described herein.

In another aspect, there is provided an isolated monoclonal antibody obtained from the hybridoma as described herein.

In another aspect, there is provided a composition comprising the isolated antibody as described herein.

In one embodiment, the composition is a therapeutic composition comprising a pharmaceutically acceptable carrier.

In another aspect, there is provided a transgenic animal comprising B-cells encoding an antibody for a target antigen, and wherein the animal has been genetically modified to express a variant antigen that is structurally related to the target antigen.

In one embodiment, the transgenic animal is a transgenic mouse.

In one specific embodiment, the B-cells in the transgenic mouse have been genetically modified to encode the antibody for the target antigen.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. Recruitment of anergic cells into GC requires higher foreign antigen density. (A) Construction of parallel groups of hematopoietic chimeras and analysis of their spleen by: (B) flow cytometry of all B cells (left) or CD45.1+SW_{HEL} B cells (right) (n=14 per group; mean±(SEM); or (C) immunohistology, showing localisation of SW_{HEL} B-cells (green), other B-cells (IgD, blue) and T-cells (CD3, magenta). (D) Relative abundance of self HEL^{3X} on mouse red blood cells (MRBCs) from mHEL^{3X}-transgenic or non-tg mice and on foreign sheep red blood cells (SRBCs) conjugated with 0 (none), 0.1 μg/mL (low) or 10 μg/mL (high) HEL^{3X}. (E) Timing of chimera immunizations. (F) Total GC cells per spleen. (G) Percentage of SW_{HEL} cells among GC B-cells. NS P>0.05, **P<0.01, ***P<0.001 by Student's t-test. Data points represent one chimera (two experiments, 16-26 chimeras in each).

FIG. 2. Gating strategy used to identify subsets of SW_{HEL} B-cells in the bone marrow.

FIG. 3. Gating strategy used to identify subsets of SW_{HEL} B-cells in the spleen.

FIG. 4. SW_{HEL} B-cells become anergic in response to self-HEL^{3X} displayed at low density on the surface of other hematopoietic and non-hematopoietic cells. (A) Schematic of the UBC:mHEL^{3X} transgene integrated at the Rosa26 locus. (B) Staining for cell surface HEL^{3X} on leukocytes and RBC from transgenic (mHEL^{3X}-Tg) or wild-type non-transgenic (non-tg) B6 mice. (C) Frequency of the indicated subsets of SW_{HEL} B-cells among all leukocytes in the bone marrow and spleen of chimeric mice reconstituted with 80% SW_{HEL} Rag1^{-/-} CD45.1 and 20% B6 non-tg or B6 mHEL^{3X}-tg bone marrow. Data points are for individual chimeras; n=3 non self-reactive SW_{HEL} and n=11 for self-reactive SW_{HEL}. (D, E) Frequency of the indicated subsets of SW_{HEL} B-cells among all leukocytes (D), or among that subset of total B220+ cells (E), in the bone marrow and spleen of chimeric mice reconstituted with 80% SW_{HEL} Rag1^{-/-} CD45.1 and 20% mHEL^{3X}-tg marrow (self-reactive SW_{HEL}; n≥11) or 45% SW_{HEL} Rag1^{-/-} CD45.1 and 55% non-tg marrow (Non self-reactive SW_{HEL}; n≥9). (F) Cell surface IgM mean fluorescence intensity (MFI), in the bone marrow and spleen of chimeras as in (D). For bone marrow N=9 per group. For spleen N=12 for non self-reactive SW_{HEL} and N=11 for self-reactive SW_{HEL}. (F, G) CD86 induction (F) and proliferation (G), gated on B220⁺CD45.1⁺HEL⁺ SW_{HEL} spleen cells from separate chimeras marrow transplanted as in (D) cultured with the indicated stimuli. N=5 per group. (H) Turnover of immature (CD45.1⁺HEL⁺CD93⁺) or mature (CD45.1⁺HEL⁺CD93⁻) SW^{HEL} B-cells in the spleen in chimeras described in (D) given BrdU in the drinking water for the indicated times. n=3 per group. Data points represent one mouse. NS P>0.05, *P<0.05, **P<0.01, ***P<0.001, ****P<0.0001. Student's t-test. Data pooled from at least two independent experiments.

FIG. 5. Expression of antigen receptors on the surface of SW_{HEL} B-cells at different stages of maturation. (A) Representative surface histograms of HEL^{3X} (0.14 μM) binding and cell surface IgD and (B, C) mean fluorescent intensity (MFI) on the indicated subsets of SW_{HEL} B-cells in individual chimeric mice, relative to the mean of the mature non-self-reactive B-cells in all mice in the same experiment, and arithmetic means. Data points represent one mouse. n=11-12 per group. Data pooled from two independent experiments. NS P>0.05, *P<0.05, **P<0.01, ****P<0.0001. Student's t-test.

FIG. 6. Representative flow cytometric profiles and gates applied to spleen cells from mice with non self-reactive (A) or self-reactive (B) SW_{HEL} B-cells, immunized with unconjugated SRBC on day 0 and with SRBC conjugated to 10 μg/mL DEL on day 11, with splenocytes analyzed on day 15.

FIG. 7. Effects of self- and foreign-antigen binding on SW_{HEL} B-cells 4 days after recruitment into GC reactions. Mice were first immunized with unconjugated SRBC to initiate GC reactions and boosted with DEL-SRBC 11 days after to activate SW_{HEL} cells and their spleens analyzed 4 days following DEL-SRBC immunization. (A) Self (HEL^{3X}-top) and foreign (DEL-bottom) protein sequences. Boxed amino acids contact the amino acid heavy chain residues listed in italics. (B) Representative immunohistological microscopy of the spleen showing IgD (blue) marking follicular mantles around GCs, CD3 (magenta) marking the T-cell zone, CD16/CD32 (white) marking follicular dendritic cells in the GC light zone, and HEL binding cells in green. (C) Total numbers of the B220⁺Fas⁺CD38⁻CD45.1⁺CD45.2⁻SW_{HEL} GC B-cells per spleen from the quadrants outlined in FIG. 2. N=6 per group. (D) Cell surface IgG1 MFI of the B220⁺Fas⁺CD38⁻CD45.1⁺CD45.2⁻SW_{HEL} GC B-cells from the quadrants outlined in FIG. 8. n=10 per group. (E,F) SW_{HEL} GC B-cells were sorted using the quadrants shown in FIG. 8 into cells with high or low binding to 0.14 μM HEL^{3X}, regardless of switched status, and single cells sequenced. Graphs show % of cells with substitutions at each H-chain amino acid in single SW_{HEL} GC cells with high (E) or low (F) self-binding, in single cell sorted non self-reactive (black) and self-reactive (red) GC SW_{HEL} B-cells. Data points represent one mouse. Data pooled from at least two independent experiments. NS P>0.05, *P<0.05, ****P<0.0001. Student's t-test.

FIG. 8. Binding to similar foreign and self-antigens triggers rapid mutation away from self. (A) Timing of chimera immunizations. (B) Total GC cells per spleen and percent SW_{HEL} cells among GC cells in chimeras receiving DEL-SRBC or unconjugated SRBC. (C) Analysis of SW_{HEL} GC B-cells, showing percentage that bind 0.14 μM HEL^{3X} or express cell surface IgG1 (mean±(SEM), and (D) percentage non-binding cells. (E) Percentage of sorted and single-cell-sequenced SW_{HEL} GC cells with S52N or S52R mutations. NS p>0.05 **P<0.01 ****P<0.0001; Student's t-test. Data points represent one mouse. Data are from at least two independent experiments each involving 3-4 mice in DEL-SRBC groups.

FIG. 9. Association and dissociation of soluble monomeric self (HEL^{3X}) or foreign (DEL) protein binding to biotinylated HyHEL10 antibody Fab mutants immobilized onto streptavidin biosensors, as measured by bio-layer interferometry. (A,B) No detectable binding of soluble self (red) or foreign (black) antigen to immobilized (A) HyHEL10^{S52R} or (B) HyHEL10^{S52N} at a concentration of 5 μM. (C,D) Association and dissociation of monomeric self (C) or foreign (D) antigen binding to immobilized HyHEL10 mutant variants. Soluble antigen was run over immobilized HyHEL10 Fab at four different concentrations to obtain global fits of binding kinetics for each interaction using the BLItz Pro 1.2 software (ForteBio). The utilized concentrations of antigen differed for each HyHEL10 variant, but all fell within a range between 5 nM (lowest foreign molarity used for HyHEL10^{I29F/S52T/Y53F}) to 5 μM (highest self-molarity used for HyHEL10^{I29F}, HyHEL10^{I29F/Y53F} and HyHEL10^{I29F/52T/Y53F}). Affinity binding constants (K_{A}, M⁻¹) and equilibrium dissociation constants (K_{D}, M) are also shown for each interaction.

FIG. 10. Self-reactive cells follow distinct mutational trajectories in order to lose self-binding capacity, leading to optimal affinity against foreign antigen. Chimera immunization timepoints were to: (A) to synchronize recruitment of SW_{HEL} B-cells into established GCs; or (B) to recruit SW_{HEL} B-cells into GCs from the outset. SW_{HEL} GC B-cells were single-cell sequenced. Dashed lines show affinity of unmutated (WT) antibody for self and foreign proteins. Circles show the affinity of recurring mutant antibodies for self and foreign proteins, with area denoting the percentage of SW_{HEL} B-cells with the indicated mutation. Red circles indicate mutations more frequent in self-reactive SW_{HEL} B-cells and blue circles represent mutations more frequent in the non-self-reactive SW_{HEL} B-cells. Data are from one experiment, representative of two, each involving 2-3 mice per group at each timepoint.

FIG. 11. Details of SW_{HEL} cell frequencies and mutations for individual mice and individual cells for the experiment shown in FIG. 3A. Mice were first immunized with unconjugated SRBC to initiate GC reactions and boosted with DEL-SRBC to activate SW_{HEL} cells and their spleens analyzed 4, 7 and 11 days following DEL-SRBC immunization as shown in FIG. 10A. (A) Total GC cells (B220⁺Fas⁺CD38⁻) per spleen from individual mice at the indicated timepoints. (B) Total SW_{HEL} GC (B220⁺Fas⁺CD38⁻CD45.1⁺CD45.2⁻) per spleen from individual mice at the indicated timepoints. (C) Total SW_{HEL} GC per spleen that do not bind to HEL^{3X} at 0.14 μM from individual mice at the indicated timepoints. (D) Total SW_{HEL} GC per spleen bearing I29F mutations from individual mice at the indicated timepoints. (E) The percentage of CD45.1⁺SW_{HEL} cells among total GC cells at the indicated timepoints. (F) The percentage of CD45.1⁺SW_{HEL} cells bearing I29F mutations at the indicated timepoints. (G) Summary of mutations at H-chain I29, S52, Y53, and Y58 in individual sorted SW_{HEL} cells. Colour coding denotes the consequence of each mutation for self-affinity. Each column represents a single cell, each row denotes whether that cell has a mutation at the indicated amino acid position, and all the cells from a single mouse are grouped within red or black boxes. Data points represent one mouse. NS P>0.05, *P<0.05. Student's t--test. Data in A-F pooled from two independent experiments. Data in (G) are from one experiment, representative of two, each involving 2-3 mice per timepoint.

FIG. 12. Details of SW_{HEL} cell frequencies and mutations for individual mice and individual cells for the experiment shown in FIG. 10B. Mice were immunized with DEL conjugated SRBC on days 0 and 4 and their spleens analyzed 15 days following first DEL-SRBC immunization as shown in FIG. 10B. (A) Total GC cells (B220⁺Fas⁺CD38⁻) per spleen from individual mice. (B) Total SW_{HEL} GC (B220⁺Fas⁺CD38⁻CD45.1⁺CD45.2⁻) per spleen from individual mice. (C) Total SW_{HEL} GC per spleen that do not bind to HEL^{3X} at 0.14 μM from individual mice. (D) Total SW_{HEL} GC per spleen bearing I29F mutations from individual mice. (E) The percentage of CD45.1⁺SW_{HEL} cells among total GC cells. (F) The percentage of CD45.1⁺SW_{HEL} cells bearing I29F mutations. (G) Ratio of SW_{HEL} GC cells that preferentially bind foreign (DEL) over self (HEL^{3X}) in individual mice. (H) Representative flow cytometric analysis of binding to self (0.14 μM HEL^{3X}) and cell surface IgG1 or foreign (DEL) proteins, gated on B220⁺Fas⁺CD38⁻CD45.1⁺CD45.2⁻ self-reactive or non self-reactive SW_{HEL} GC B-cells. Mean±SEM displayed for each quandrant. (I) Summary of mutations at H-chain I29, S52, Y53, and Y58 in individually sorted and sequenced SW_{HEL} cells. Colour coding denotes the consequence of each mutation for self-affinity. Each column represents a single cell, each row denotes whether that cell has a mutation at the indicated amino acid position, and all the cells from a single mouse are grouped within red or black boxes. (J) The percentage of SW_{HEL} cells with substitutions at each H-chain amino acid. Data points represent one mouse. NS P>0.05, *P<0.05 **P<0.01. Student's t-test. Data in (A-F) and (H-J) are pooled from at least two independent experiments, each involving 3-5 mice per group. Data in (G) represents one experiment.

FIG. 13. IgG1 SW_{HEL} memory response. (A-C) Mice were first immunized with unconjugated SRBC to initiate GC reactions and boosted with DEL-SRBC to activate SW_{HEL} cells and their spleens analyzed 4, 7 and 11 days following DEL-SRBC immunization as shown in FIG. 3A. (A) Total IgG1 switched memory cells (B220⁺Fas⁻CD38⁺IgG1⁺) per spleen from individual mice at the indicated timepoints. (B) Total SW_{HEL} IgG1 switched memory cells (B220⁺Fas⁻CD38⁺IgG1⁺CD45.1⁺CD45.2⁻) per spleen from individual mice at the indicated timepoints. (C) Total SW_{HEL} IgG1 switched memory cells per spleen that do not bind to HEL^{3X} at 0.14 μM from individual mice at the indicated timepoints. (D-H) Mice were immunized with DEL-SRBC on days 0 and 4 and analyzed day 15 as shown in FIG. 10B. (D) Total IgG1 switched memory cells (B220⁺Fas⁻CD38⁺IgG1⁺) per spleen. (E) Total SW_{HEL} IgG1 switched memory cells (B220⁺Fas⁻CD38⁺IgG1⁺CD45.1⁺CD45.2⁻) per spleen from individual mice. (F) Total SW_{HEL} IgG1 switched memory cells per spleen that do not bind to HEL^{3X} at 0.14 μM from individual mice. (G) Ratio of SW_{HEL} IgG1 memory cells that preferentially bind foreign (DEL) over self (HEL^{3X}) in individual mice. (H) Representative flow cytometric analysis of relative binding of individual SW_{HEL} IgG1 memory cells to soluble monomeric self (HEL3^{X}) and foreign (DEL) proteins. Mean±SEM displayed for each gate. Data points represent one mouse. NS P>0.05, *P<0.05 **P<0.01 ***P<0.001. Student's t-test. Data in (A-F) are pooled from at least two independent experiments, each involving 2-5 mice per group. Data in (G) and (H) represents one experiment.

FIG. 14. Early plasmablast (PB) and serum antibody response. (A-H) Mice were first immunized with unconjugated SRBC to initiate GC reactions and boosted with DEL-SRBC to activate SW_{HEL} cells and their spleens analyzed 4 days following DEL-SRBC immunization as shown in FIG. 8A. (A) Total PBs (B220⁺Fas⁻CD38⁻IgD⁻TACI⁺CD138⁺) per spleen. (B) Total SW_{HEL} PBs (B220⁺Fas⁻CD38⁻IgD⁻TACI⁺CD138⁺CD45.1⁺CD45.2⁻) per spleen. (C) Total SW_{HEL} PBs per spleen that have retained binding to self (HEL^{3X}) at 0.14 μM. (D) The percentage of CD45.1⁺SW_{HEL} PBs among total PBs. (E) The percentage of SW_{HEL} PBs that have retained binding to self. (F) Representative histograms of SW_{HEL} PBs showing self-binding. (G) Serum antibody ELISA showing self-binding Igκ. (H) Serum antibody ELISA showing self and foreign (DEL) binding IgG1. (I) Serum antibody ELISA showing self and foreign binding IgG1 from individual mice immunized with DEL-SRBC on days 0 and 4 and then harvested 15 days post DEL SRBC as shown in FIG. 10B. Data points represent one mouse. NS P>0.05, *P<0.05 ****P<0.0001. Student's t-test. Data are representative of one experiment, with 3-5 mice per group.

FIG. 15. Structural basis of mutation away from self. X-ray crystallographic structures of (A) unmutated HyHEL10 in complex with HEL and (B) HyHEL10^{I29F,S52T,Y53F} triple mutant antibody in complex with DEL. (C) Overlay of both structures showing the structural rearrangement of the CDR1 loop caused by the I29F mutation (1) and the complementary structural adjustments of positions 52 and 53 in the CDR2 loop to exploit the Leu75Ala pocket in the foreign antigen (2).

FIG. 16. X-ray crystallographic structure of unmutated HyHEL10 in complex with HEL (A) and HyHEL10^{129F} mutant antibody in complex with DEL (B).

FIG. 17. Resolution of antigenic mimicry by rapid evolution of polyclonal self-reactive B-cells to lose self-binding and retain foreign specificity. (A) Flow cytometric analysis of spleen B-cells pooled from mice with a normal antibody repertoire expressing HEL^{3X} as a ubiquitous membrane self-antigen. B-cells with anergic IgD⁺IgM^{low} phenotype were gated, and further subdivided to sort the subset staining brightly with 0.14 μM monomeric HEL^{3X}, comprising 0.5% of all B-cells. The percentage of parent cells within the indicated gates is shown. Sorted polyclonal HEL^{3X} binding anergic B-cells (CD45.2⁺) were mixed with unselected spleen B-cells (CD45.1⁺) to comprise 0.5% of the mixture, and transferred by intravenous injection together with T-cells into mHEL^{3X} tg Rag1^{-/-} mice so that the transferred B-cells could be tracked by flow cytometry. The recipient mice were immunized with foreign DEL-SRBC on days 0 and 4, boosted with DEL conjugated to horse red blood cells (HRBCs) on day 14, and splenocytes analyzed by flow cytometry on day 20. (B) CD45.2⁺ B-cells, derived from the sorted polyclonal HEL^{3X}-binding anergic B-cell inoculum, were enumerated by flow cytometry as a percentage of all B-cells, either in the transferred mixture (left), among all GC B-cells (B220⁺Fas⁺CD38⁻) on day 20 (middle), and among the subset of GC B-cells binding to foreign DEL-biotin on day 20 (right). Representative flow cytometric plots from day 20 are shown. Numbers show mean percentage and s.e.m of cells in each gate. (C) GC cells on day 20 were stained first with 0.14 μM monomeric HEL^{3X} and then with DEL-biotin, and analyzed by flow cytometry to enumerate the percentage of CD45.2⁺ or CD45.1⁺ GC cells that selectively bound the foreign or self lysozymes. Data points represent one mouse. NS P>0.05 ****P<0.0001. Student's t-test. Data shown from one experiment, representative of two, with 4 mice per group.

FIG. 18. Autoantibody redemption can still occur against a flexible epitope. (A) Root Mean Square Deviation (RMSD) simulation of the listed HEL variants (B) Percentage of HyHEL10 cells amongst GC B-Cells from mice with or without self-HEL3× immunised with DEL or HEL2X-Flex at the indicated timepoints after antigen exposure. Data points represent one mouse. (C) Mutational trajectory of HyHEL10 B cells towards self and foreign antigens. Circles show the affinity of recurring mutant antibodies for self and foreign proteins, with area denoting the percentage of HyHEL10 B-cells with the indicated mutation. Data representative of 3 independent experiments with 3-4 mice per group.

FIG. 19. Autoantibody redemption can still occur against a flexible epitope. (A) HEL^{3X}, DEL, HEL^{2X}-rigid and HEL^{2X}-flex protein sequences. Amino acids in blue are different from the HELWT protein. Boxed amino acids contact the amino acid heavy chain residues listed in italics. (B) Root Mean Square Deviation (RMSD) during HEL^{WT} simulation. (C) RMSDs during HEL^{R73E} simulation. (D) RMSDs during HEL^{R21Q} simulation. (E) RMSDs during HEL^{C76S,C94S} simulation. (F) Total GC cells (B220⁺Fas⁺CD38⁻) per spleen from individual mice at 15 days post antigen exposure. (G) Total HyHEL10 GC (B220⁺Fas⁺CD38⁻CD45.1⁺CD45.2⁻) per spleen from individual mice at 15 days post antigen exposure. (H) Total serum IgK from chimeras harvested on day 15 following antigen exposure. (I) Total HEL^{3X} binding serum IgK from chimeras harvested on day 15 following antigen exposure. (J) Total DEL binding serum IgK from chimeras harvested on day 15 following antigen exposure. (K) Total HEL^{2X}-Flex binding serum IgK from chimeras harvested on day 15 following antigen exposure. (L) Average numbers of synonymous mutations per GC HyHEL10 B cell. (M) Average numbers of non-synonymous mutations per GC HyHEL10 B cell. **P<0.01 Student's t-test. Data points represent one mouse. (N) The percentage of GC HyHEL10 B cells with substitutions at each H-chain amino acid. Data represents 2 independent experiments per timepoint with 1-2 mice per group.

FIG. 20. Autoantibody redemption can still occur against a flexible epitope. Summary of mutations at H-chain L4, I29, S31, Y33 S52, Y53, S56 and Y58 in individual sorted HyHEL10 GC B cells. Color coding denotes the consequence of each mutation for DEL and HEL^{2X}-flex affinity. Each column represents a single cell, each row denotes whether that cell has a mutation at the indicated amino acid position, and all the cells from a single mouse are grouped within red or black boxes. Data representative of 3 independent experiments with 3-4 mice per group.

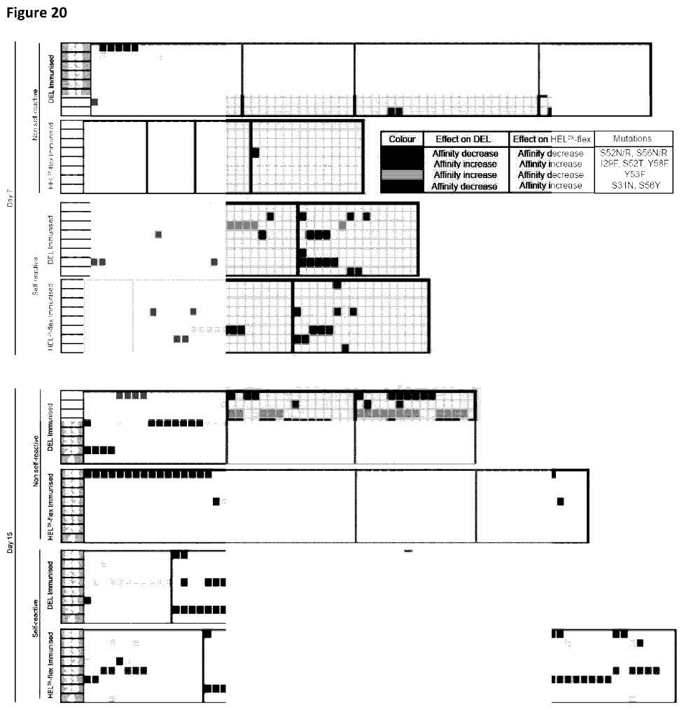

FIG. 21. Comparison of autoantibody redemption between a flexible epitope and a rigid variant of the same epitope. (A) Percentage of HyHEL10 cells amongst GC B-Cells from mice with or without self-HEL^{3X} immunised with HEL^{2X}-Flex or HEL^{2X}-rigid at the indicated timepoints after antigen exposure. (B) Percentage of HyHEL10 cells amongst IgG1 memory B-Cells from mice with or without self-HEL^{3X} immunised with HEL^{2X}-Flex or HEL^{2X}-rigid at the indicated timepoints after antigen exposure. (C) Average number of synonymous and non-synonymous mutations per HyHEL10 B cell. *P<0.05, **P<0.01, ****P<0.0001 by Student's t-test. Data points represent one mouse. (D) Mutational trajectory of HyHEL10 B cells to self and foreign antigen. Circles show the affinity of recurring mutant antibodies for self and foreign proteins, with area denoting the percentage of HyHEL10 B-cells with the indicated mutation. Data pooled from 2 experiments per timepoint with 2-3 mice per group.

FIG. 22. Comparison of autoantibody redemption between a flexible epitope and a rigid variant of the same epitope. (A) Total GC cells (B220⁺Fas⁺CD38⁻) per spleen from individual mice at the indicated timepoints post antigen exposure. (B) Total HyHEL10 GC (B220⁺Fas⁺CD38⁻CD45.1⁺CD45.2⁻) per spleen from individual mice at 15 days post antigen exposure. (C) Total IgG₁ memory cells (B220⁺Fas⁻CD38⁺IgG₁⁺) per spleen from individual mice at the indicated timepoints post antigen exposure. (D) Total HyHEL10 IgG₁ memory cells (B220⁺Fas⁻CD38⁺IgG₁⁺ CD45.1⁺CD45.2⁻) per spleen from individual mice at the indicated timepoints post antigen exposure. (E) Mutational trajectory of HyHEL10 B cells relative to HyHEL10 starting affinity. Circles show the affinity of recurring mutant antibodies for self and foreign proteins, with area denoting the percentage of SW_{HEL} B-cells with the indicated mutation. (F) Total serum IgG₁ from chimeras harvested on day 15 following antigen exposure. (G) Total HEL^{2X}-Rigid binding serum IgG₁ from chimeras harvested on day 15 following antigen exposure. (H) Total HEL^{3X} binding serum IgG₁ from chimeras harvested on day 15 following antigen exposure. *P<0.05,**P<0.01 Student's t-test. Data points represent one mouse. Data representative of 2 independent experiments per timepoint with 2-3 mice per group.

FIG. 23. Comparison of autoantibody redemption between a flexible epitope and a rigid variant of the same epitope. (A) The percentage of HyHEL10 cells with substitutions at each H-chain amino acid at the indicated timepoints post antigen exposure. (B) Summary of mutations at H-chain L4, I29, S31, Y33, S56 and Y58 in individual sorted HyHEL10 cells. Each column represents a single cell, each row denotes whether that cell has a mutation at the indicated amino acid position, and all the cells from a single mouse are grouped within red or black boxes.

Data representative of 2 independent experiments per timepoint with 2-3 mice per group.

FIG. 24. Rigid and floppy antigens, generated by a C76S mutation, which breaks a structural disulphide bridge, permits a different loop conformation (thick black arrow) which facilitates tight binding to different antibody surfaces (antibody surface 1 and antibody surface 2).

Key to the Sequence Listing

SEQ ID NO: 1 Amino acid sequence for wild-type HEL protein.

SEQ ID NO: 2 Amino acid sequence for a mutant flexible HEL protein.

SEQ ID NO: 3 Amino acid sequence for a mutant rigid HEL protein.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in immunology, immunohistochemistry, protein chemistry, biochemistry and chemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of immunoglobulins, antibodies and fragments thereof herein may be further understood by the discussion in Kabat, 1987 and/or 1991, Bork et al., 1994 and/or Chothia and Lesk, 1987 and/or 1989 or Al-Lazikani et al., 1997.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

As used herein, the term “target” shall be understood to refer to an antigen against which it is desired to produce an antibody. A target antigen in one embodiment is a foreign antigen. As used herein, the term “antigen” shall be understood to mean any composition of matter against which an immunoglobulin response (e.g., an antibody response) could potentially be raised. Exemplary antigens include proteins, peptides, polypeptides, carbohydrates, phosphate groups, phosphor-peptides or polypeptides, glyscosylated peptides or peptides, etc.

Functionally important vaccine targets are often highly disordered or surrounded by flexible loops. In one embodiment, the target antigen may be a conformationally-flexible antigen. Suitable examples of conformationally-flexible antigens include, but are not limited to, HIV envelope protein, circumsporozoite protein (CSP), merozoite surface protein 2(MSP2) and GAD65. In one example, the Root Mean Square Deviation of the target antigen is at least 0.5, 1, 2, 3, 4, 5 Angstroms. There are techniques known in the art to generate a conformationally-flexible antigen. For example, an antigen may be made conformationally-flexible by mutation or by denaturation such as by varying temperature or pH or by the addition of adjuvants.

As used herein, the term “affinity” shall be understood to refer to the strength of interaction between the binding sites of the antibodies disclosed herein with the antigen. In one example described herein, strength of interaction may be determined by measuring the strength of binding reaction through affinity constants which are well known in the art. In one example, strength of binding between the immunoglobulin homodimers described herein and the antigen may be determined by measuring the equilibrium binding constant (K_{D}). The method described herein provides that affinity may be measured using any suitable techniques, including any suitable optical analytical techniques for measuring biomolecular interactions known in the art. In one example, optical analytical techniques may include: Bio-Layer Interferometry. In another example, the optical analytical technique may include: dual polarisation interferometry, static light scattering, dynamic light scattering, surface plasmon resonance, fluorescence polarisation/anisotropy, fluorescence correlation spectroscopy or nuclear magnetic resonance.

The term “basic affinity” as used herein refers to the affinity of an antibody for a target antigen prior to performing the method as described herein. As a result of performing the method described herein, the antibody will have an improved affinity for the target antigen. As understood in the art, K_{D} and affinity are inversely related. The K_{D} value relates to the concentration of antibody (for example, the amount of antibody needed for a particular experiment) and so the lower the K_{D} value (lower concentration), the higher the affinity of the antibody. Thus, an improvement or increase in affinity relates to a lower numerical K_{D} figure. For example, a decrease in K_{D} from 1×10⁻⁶ M to 1×10⁻⁷ M relates to an improvement or increase in affinity.

Accordingly, the basic affinity of the antibody prior to performing the method described herein is lower than a desired range for an intended purpose. For example, desirable antibody affinities for therapeutic antibodies may be represented by a K_{D} in the range of low nanomolar (nM) to picomolar (pM), and to the femtomolar (fM) range. Accordingly, if a desirable antibody affinity is in the low nM range, then a basic affinity will be less than this, and may be, for example, in the μM to high nM range. Thus, in one embodiment, the basic affinity of the antibody to the target antigen is low affinity relative to a desired affinity. In one embodiment, low affinity relative to a desired affinity may be at least 10-fold, at least 100-fold, at least 1000 fold, or at least 10,000-fold lower affinity than the desired affinity. In one embodiment, the basic affinity for the target antigen is a K_{D} of about 10⁻⁶ M to about 10⁻⁸ M. Thus, as used herein the term “improved affinity” refers to the affinity of the antibody for the target antigen after performing the method as described herein. In certain embodiments, the improved affinity will be at least 10-fold, at least 100-fold, at least 1000-fold or at least 10,000-fold or more improvement over the basic affinity for the target antigen.

As used herein, the term “variant antigen that is structurally related to the target antigen” refers to an antigen that has a closely related structure to the target antigen, such that prior to performing the method as described herein, the antibody on the B-cell cross-reacts with both the target antigen and the variant antigen. In one embodiment, the variant antigen is a self-antigen. For example, in one embodiment, the antibody has a basic specificity (i.e. the specificity of the antibody prior to performing the method as described herein) that is represented by a less than 10-fold difference in binding affinity for the target antigen compared to the binding affinity for the variant antigen. In another embodiment, the basic specificity may be a binding affinity that is about 10⁻⁶ to about 10⁻⁷ for both the target antigen and the variant antigen.

For example, for a peptide or polypeptide antigen, the variant antigen that is structurally related to the target antigen may comprise one or more amino acid residue variants compared to the target antigen that result in a change in, for example, the primary, secondary or tertiary structure of the molecule. In certain embodiments, the variant residue may be in a linear epitope, a surface that contacts the antibody, or a conformational epitope, or in any other region of a peptide or polypeptide that affects the structure of the molecule. For carbohydrate antigens, there are techniques known in the art to structurally modify carbohydrate antigens. In the context of transgenic animals, modification of carbohydrate antigens may be achieved through the modification or deletion of, for example, enzymes in carbohydrate biosynthesis pathways.

Antibody specificity is the degree to which an antibody differentiates between two antigens. A simple approach measures the relative binding affinities of an antibody to the target antigen and one or more variant antigens. Discrimination depends on the range of variants to which the antibody binds, on the binding affinity, and on the stringency of the conditions under which the assay is conducted. Such binding specificity may be determined by methods well known in the art, such as ELISA, immunohistochemistry, immunoprecipitation, Western blots and flow cytometry using transfected cells expressing the antigen.

As used herein, the term “basic specificity” refers to the specificity of the antibody for binding the target antigen and variant antigen prior to performing the method as described herein. Thus, the term “basic specificity” refers to an antibody that is cross-reactive with both the target antigen and variant antigen.

As used herein, the term “improved specificity” refers to the specificity of the antibody after performing the method described herein. Accordingly, the isolated B-cells expressing antibody with improved specificity will typically exhibit increased affinity for the target antigen and a decreased affinity to the variant antigen as compared to the basic specificity of the antibody. In one embodiment, improved specificity for the target antigen is an increase in the ratio of the affinity for the target antigen to the affinity for the variant antigen by at least 10-fold, or at least 100-fold, or at least 1000-fold, or at least 10,000-fold.

### EXAMPLES

### Example 1. Mice

All mice used in the experiments were bred at Australian BioResources and held at the Garvan Institute of Medical Research in specific pathogen-free environments. The Garvan Animal Ethics Committee approved all mice protocols and procedures. C57BL/6 (non-transgenic) mice were purchased from the Australian BioResources (Moss Vale, New South Wales). HyHEL10-transgenic (SW_{HEL}) mice have been described previously (Phan et al., 2003 J. Exp. Med. 197, 845-860 (2003)). These mice carry a single copy V_{H}10 anti-HEL heavy chain variable region coding exon targeted to the endogenous Igh^{b} allele plus multiple copies V_{H}10-κ anti-HEL light chain transgene. SW_{HEL} mice on a CD45.1 congenic (Ptprc^{a/a}) C57BL/6 background were also homozygous Rag1^{-/-}, which prevented endogenous Ig variable region gene rearrangements so that all B-cells expressed the HyHEL10 B-cell receptor (BCR).

HEL^{3X} is an R21Q, R73E, and D101R triply mutated HEL protein. HEL^{3X} binds to HyHEL10 with relatively low affinity of Kₐ=1.19×110⁷ M⁻¹. Transgenic mice on the C57BL/6 background expressed HEL^{3X} as an integral cell surface protein, by addition at the C-terminus of the transmembrane segment and cytoplasmic tail of the H2K^{b} class I major histocompatibility protein. The transgene was controlled by the human ubiquitin C (UBC) promoter, resulting in membrane bound HEL^{3X} expression on the surface of all nucleated and anucleate cells. HEL^{3X} membrane expression was confirmed using flow cytometric binding to HyHEL9 on RBC and WBC.

HEL^{R21Q,R73E,C76S,C94S} (HEL^{2X}-flex) is mutant version of HEL, designed with four mutations compared to mature wild-type HEL. WT HEL amino acid residues R21 and R73 both have positively charged side chains and are contained within the binding epitope of HyHEL10. The R21Q and R73E found in HEL^{2X}-flex both cause unfavourable charge reversals in the side chains of these residues and thus directly decrease HyHEL10 binding affinity. In contrast, mutations C76S and C94S remove a cysteine disulphide bond from the HEL protein, causing structural flexibility in the HEL protein in the region containing the HyHEL10 binding epitope.

HEL^{R21Q,R73E} (HEL^{2X}-rigid) is another variant that has the same contact residue mutations as HEL^{2X}-flex but has the disulfide bond, giving it a more rigid conformational structure.

### Example 2. Bone Marrow Chimeras

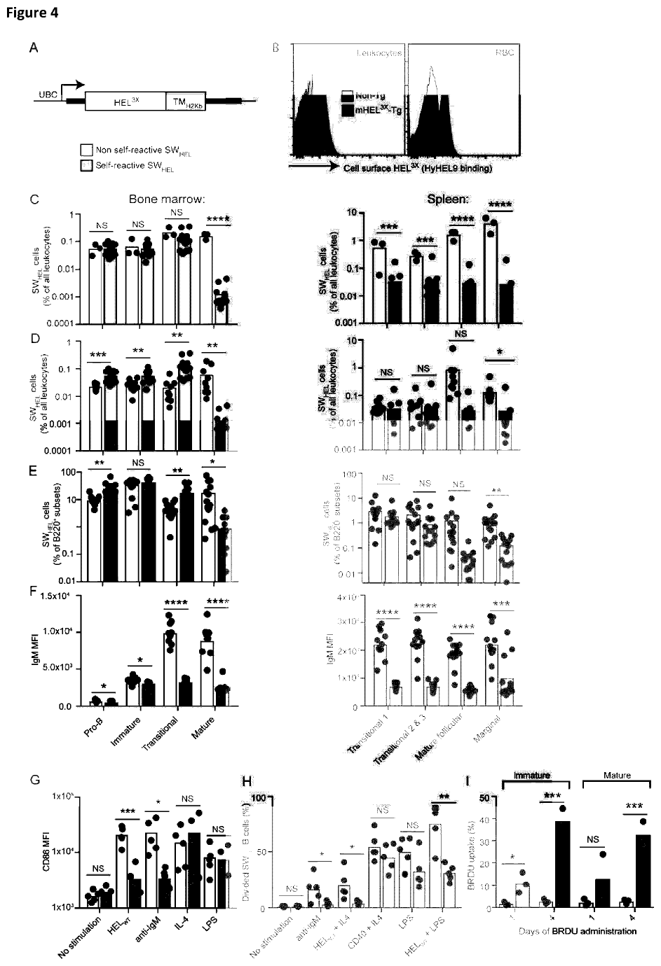

Recipient mice of 8-12 weeks of age were lethally irradiated (2×425 cGy) using an XRAD 320 Biological Irradiator (Precision X-Ray, North Branford, Conn., UA). Femoral, humeral, and tibial bone marrow cells were aspirated into B-cell medium (BCM) comprising RPMI (Gibco, Carlsbad, Calif., USA) with 10% heat-inactivated Fetal Calf Serum (FCS) (Gibco), 2 mM L-glutamine, 100 U/mL penicillin RPMI media (Gibco). Fifteen hours after irradiation recipient mice were transplanted with an intravenous injection of 5-10×10⁶ bone marrow cells. For mHEL^{3X} transgenic (CD45.2⁺) recipients, injected bone marrow cells were 80% of SW_{HEL}.Rag1^{-/-} (CD45.1⁺) origin and 20% of mHEL^{3X} transgenic (CD45.2⁺) origin. As previously observed, CD45.2⁺ pre-B-cells lacking pre-rearranged Igh and Igk genes proliferated more than their SW_{HEL} (CD45.1⁺) Ig transgenic counterparts, so that only ˜5% of B220+ in chimeric recipients were CD45.1⁺ anti-HEL B-cells. Initially (FIG. 4), chimeras produced in control non-transgenic C57BL/6 (CD45.2⁺) mice also used a bone marrow cell mix that was 80% SW_{HEL}.Rag1−/− (CD45.1⁺) and 20% recipient-matched (wild-type CD45.2⁺). Subsequently, control non-transgenic mice were reconstituted with less (45%) SW_{HEL}.Rag1^{-/-} (CD45.1⁺) and more (55%) wild-type (CD45.2⁺) bone marrow, so that comparable frequencies of anti-HEL B-cells in the transitional subset of the spleen were obtained in chimeras irrespective of self-antigen (mHEL^{3X}) expression. Chimeras were analyzed 8-14 weeks after reconstitution.

### Example 3. Immunohistology

Five-to-seven-micron sections were cut using a Leica CM1900 cryostat, fixed in acetone and blocked with 30% normal horse serum. To stain HyHEL10 B-cells, sections were incubated with 200 ng/mL HEL (Sigma, St Louis, Mo., USA), polyclonal rabbit anti-HEL sera, and anti-rabbit-IgG-FITC (Rockland Immunochemicals, Pottstown, Pa., USA). T-cells were stained with anti-CD3-biotin (eBiosciences, San Diego, Calif., USA) and streptavidin-AlexaFlour 555 (Invitrogen, Carlsbad, Calif., USA). Follicular B-cells were stained with anti-IgD-AlexaFlour 647 (Biolegend, San Diego, Calif., USA). For GC analysis, GCs were stained with anti-CD16/CD32-PE (BD Pharmingen, San Diego, Calif., USA) and CD3-biotin stains were followed by streptavidin-BV421 (BD Pharmingen). Stained tissue sections were imaged using a Zeiss Leica DM5500 microscope. AxioVision software was used for image capture and Adobe Photoshop used to compile the images. Images shown are 200× magnification.

### Example 4. In Vitro Cultures

B-cell responses in vitro were determined by culturing fresh splenocytes overnight at 37° C. in BCM following red-blood-cell lysis. B-cells were stimulated with one of the following additives: HEL^{WT} (200 ng/mL), LPS (2.5 μg/mL, Sigma), recombinant mouse IL-4 (10 ng/mL, R&D Systems, Minneapolis, Minn.) or anti-IgM mAb (5 μg/mL, Southern Biotech, Birmingham, Ala., USA). Cells were then surface stained to detect the upregulation of CD86 by flow cytometry.

Alternatively, fresh splenocytes were resuspended at 2×10⁷ cells/mL in PBS containing 5% FCS and CFSE labeled with a final concentration of 11 μM for 5 minutes at room temperature. Cells were washed in PBS containing 5% FCS and then resuspended in BCM. B-cells were stimulated with anti-CD40 mAb (5 μg/mL, BD Pharminigen), LPS, anti-IgM mAb, IL-4, HEL^{WT} plus LPS, anti-CD40 mAb plus IL-4, HEL^{WT} plus IL-4 or anti-IgM mAb plus IL-4. Cells were cultured for 3 days at 37° C. proliferation was assessed as loss of CFSE staining via flow cytometry.

### Example 5. Recombinant HEL Proteins

Purified HEL^{WT} (SEQ ID NO: 1) was purchased from Sigma-Aldrich. Recombinant HEL^{3X} and DEL proteins were made as secreted proteins in Pichia pastoris yeast (Invitrogen) and purified from culture supernatants by ion exchange chromatography as previously described (Phan et al. 2003; Paus et al. 2006; Chan et al. 2012 Immunity 37, 893-904 (2012).

In order to generate the HEL^{R21Q,R73E,C76S,94S} (HEL^{2X}-flex, SEQ ID NO: 2) and HEL^{R21Q,R73E} (HEL^{2X}-rigid, SEQ ID NO: 3) variants, DNA encoding the HEL^{2X}-flex amino acid sequence or the HEL^{2X}-rigid amino acid sequence was cloned into the pCEP4 expression vector. Transient expression was then carried out in the Expi293™ transient expression system (Thermo Fisher Scientific) following the manufacturer's instructions.

Affinity chromatography medium was prepared by covalent coupling of anti-HEL V_{HH} domain D2L19 (De Genst et al. 2006) to CNBr-Activated Sepharose 4B (GE Healthcare).

Soluble HEL^{2X}-flex or HEL^{2X}-rigid was then purified from the culture supernatant using the D2L19-coupled sepharose followed by elution with 0.1 M Glycine pH 2.7. Purified protein was then dialysed into PBS using snakeskin 3.5 kDa MWCO dialysis tubing (Pierce) and used for conjugation to SRBC as described in Example 6 for DEL and other related antigens.

For the purposes of crystallography, DEL lacking a poly-histidine affinity tag was purified from duck eggs. Proteins were stored in PBS at 1-2.5 mg/mL at −80° C. Prior to use samples were thawed and stored at 4° C. for a maximum of 8 months.

### Example 6. Sheep Red Blood Cell (SRBC) Conjugation

HEL proteins were desalted into conjugation buffer (distilled water with 0.35 M DMannitol [Sigma] and 0.01 M Sodium Chloride [Sigma]). For this process PD-10 columns (Amersham, Piscataway, N.J., USA) were equilibrated with 30 mL Conjugation buffer. One hundred micrograms of protein was loaded onto each column and pushed through the column using 2.5 mL Conjugation buffer. For elution of the protein, 3.5 mL conjugation buffer was added and the HEL protein collected as fractions in the following volumes; 250 μL, 1000 μL, 250 μL, 250 μL, 250 μL. Protein concentrations of each 4 fraction were determined by spectrophotometry.

For conjugation, SRBC were washed in 30 mL of PBS per 6-8×10⁹ cells. Washing was performed three times by centrifugation at 2,300 rpm (1,111 g) for 5 min at 4° C. in PBS and then once in conjugation buffer. SRBC were then resuspended in a final volume of 1 mL conjugation buffer in a 50 mL Falcon tube containing 10 μg/mL of protein for conjugation, unless otherwise stated, which had first been buffer exchanged by gel filtration on PD10 columns into the conjugation buffer. The solution was mixed on a platform rocker on ice for 10 minutes. One hundred microliters of 100 mg/mL N-(3-Dimethylaminopropyl)-N-ethylcarbodimide hydrochloride (Sigma) was then added and the solution was mixed for a further 30 minutes on ice. SRBCs were then washed four times in 30 mL PBS. Confirmation of successful conjugation was performed by flow cytometric analysis of SRBC using AlexaFluor 647-conjugated HyHEL9 antibody. 2×10⁸ conjugated or unconjugated SRBC were injected into the lateral tail vein of each chimeric mouse.

### Example 7. Flow Cytometry

On the day of harvest organs were collected into BCM, cell suspensions passed through a 70 μm cell strainer (Falcon, Corning, N.Y., USA) and centrifuged 1 500 rpm (440 g) for 5 min at 4° C. Fc receptors were blocked with unlabeled anti-CD16/32 (ebioscience) before staining. To detect HEL^{3X}-binding cells, cells were stained with 2 μg/mL (0.14 μM) HEL^{3X}, followed by AlexaFluor 647-conjugated HyHEL9. Since this concentration of HEL^{3X} approximates the Ka of the unmutated HyHEL10 receptor on the B-cells, it occupied approximately half of the binding sites.

For DEL-N terminal biotinylation, DEL was conjugated to 5 M excess of NHS biotin (Sigma) at pH 6.5 overnight on ice. For panels where DEL-biotin staining was concurrently used with HyHEL9, HyHEL9 stains were followed with HEL^{4X} at 2 μg/mL to block any unbound HyHEL9 binding sites without binding to the HyHEL10 BCR. DEL-biotin staining then followed at 2 μg/mL.

Anti-IgG1-FITC (BD Pharmingen) stains were followed by 5% mouse serum before staining for other surface molecules. Cells were filtered using 35 μm filter round-bottom FACS tubes (BD Pharmingen) immediately before data acquisition on a LSR II analyzer (BD Pharmingen).

Forward- and side-scatter threshold gates were applied to remove red blood cells and debris and approximately 5-7×10⁶ events were collected per sample. Cytometer files were analyzed with FlowJo software (FlowJo LLC, Ashland, Oreg., USA).

### Example 8. BrdU Staining

BrdU staining was performed as described previously (16). Briefly mice were given drinking water shielded from light containing 0.8 mg/mL BrdU (Sigma). Spleens and bone marrow were prepared as for flow cytometry. Following surface staining cells were fixed and permeabilized, DNA denatured and then stained with anti-BrdU FITC using BrdU staining kit (BD Phaminigen) as per the manufacturer's directions.

### Example 9. Fab Expression and Purification

Mutant and wild-type HyHEL10 heavy (IgH) and wild-type HyHEL10 kappa-light (IgK) chain FAb sequences were synthesized and cloned into pCEP4 expression vector via KpnI and BamHI restriction sites. The heavy chain was C-terminally his-tagged for purification purposes. Fab arms were transiently expressed using the Expi293 Expression System (Thermo Fisher Scientific, Boston, Mass., USA) according to the manufacturer's recommendations. Lipid-DNA complexes were prepared using a 1H:2L chain ratio, as previously described (28). Fab was purified from cell culture supernatant using HisTrap FF crude columns (GE Healthcare, Little Chalfont, UK) according to the manufacturer's instructions. After dialysis against PBS Fabs were concentrated using spin filters (EMD Millipore, Billerica, Mass., USA), inspected on SDS-PAGE and their concentrations determined by spectrometry (absorbance at 280 nm).

### Example 10. Analysis of Binding Affinity

Purified HyHEL10 FAbs were buffer exchanged into PBS using equilibrated ZebaSpin columns (Thermo Fisher Scientific). FAb samples were requantified and incubated with EZ-Link NHS-PEG4-Biotinylation reagent (Thermo Fisher Scientific) at a 5:1 biotin-toprotein ratio. Free biotin was removed from the samples by repeating the buffer exchange step in a second zebaspin column equilibrated with PBS.

Affinity of interactions between biotinylated FAbs and purified lysozyme proteins (DEL, HEL^{3X}, HEL^{2X}-flex and HEL^{2X}-rigid) by Biolayer Interferometry (BLItz, ForteBio, Menlo Park, Calif., USA). Streptavidin biosensors were rehydrated in PBS containing 0.1% w/v BSA for 1 hr at RT. Biotinylated FAb was loaded onto the sensors “on-line” using an advanced kinetics protocol, and global fits were obtained for the binding kinetics by running associations and dissociations of Lysozyme proteins at a suitable range of molar concentrations. The global dissociation constant (KD) for each 1:1 FAb-lysozyme interaction was determined using the BlitzPro 1.2.1.3 software.

### Example 11. Single Cell FACS Sorting

Cell suspensions were prepared and germinal center B-cells identified as for flow cytometry. Single-cell sorting into 96-well plates (Thermo Fisher Scientific) was performed on a FACSAria or FACSAriaIII (BD Pharminigen). B-cells from each mouse were analyzed individually to ensure over-representation of one particular clone did not affect mutation analysis. The VDJH exon of the HyHEL10 heavy chain gene was amplified from genomic DNA by PCR, sequenced, and analyzed.

### Example 12. ELISA

ELISA detection of serum concentrations of IgG1 antibodies binding to HEL^{3X}/DEL were measured. High-binding plates (Corning, Corning, N.Y., USA) were coated with HEL^{3X} or DEL and bound serum antibody quantified using the same IgH chain isotype-specific secondary antibodies used for flow cytometry. Antibody levels were quantified against HyHEL10 standards.

### Example 13. Crystallography

The complex comprising DEL purified from duck eggs (isoform DEL-I) and the Fab arm of HyHEL10^{I29F,S52T,Y53F} (HH10*3) was prepared by gel filtration chromatography in which a 2:1 ratio of DEL:HH10*3 was applied to an S200 26/60 column (GE Healthcare) plumbed with 25 mM Tris (pH 8.5), 150 mM NaCl. Crystals of HH10*3-DEL and other HyHEL10 complexes were grown by hanging drop vapor diffusion whereby 2 L of protein complex (at ˜6.5 mg/mL) was combined with an equal volume of well solution comprising 100 mM sodium citrate (pH 4.75 and 17% v/v) PEG3350 (Hampton Research, Aliso Viejo, Calif., USA). Crystals grew over several weeks. Crystals were briefly swum (10 sec) in well solution doped with glycerol to ˜25% (v/v) prior to being flash frozen in N₂ (1 sec) for data collection.

### Example 14. Diffraction Data, Structure, Solution and Refinement

Diffraction data were collected at the Australian Synchrotron on beamline MX2 at 100 K. Diffraction data were indexed and integrated using iMOSFLM, the space group determined with POINTLESS, and scaling performed with AIMLESS. Structures were solved via molecular replacement using PHASER and employing PDB entries 3D9A (Fab) and 5V8G (DEL-I) as search models. Rigid-body and restrained B-factor refinement were performed with REFMAC5, part of the CCP4 suite of crystallography software. Models were inspected and compared with electron density maps, and where necessary modified, using COOT. Validation was performed using the MOLPROBITY server.

### Example 15. Quantification and Statistical Analysis

GraphPad Prism 6 (GraphPad Software, San Diego, USA) was used for data analysis. When the data were normally distributed, an unpaired Student's t-test was performed for analysis. When data was not normally distributed Welsh's correction was applied. For all tests, P<0.05 was considered as being statistically significant. Unless otherwise stated error bars represent arithmetic mean. Flow cytometric plots of multiple samples are presented as mean and standard error or mean. For all figures, data points indicate individual mice. * represents P<0.05, ** represents P<0.01, *** represents P<0.001, **** represents P<0.0001.

### Example 16. Results

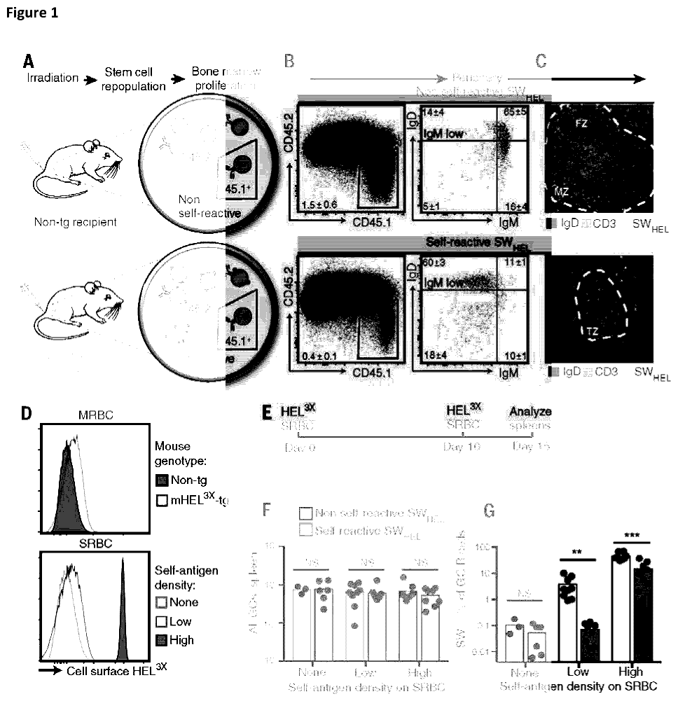

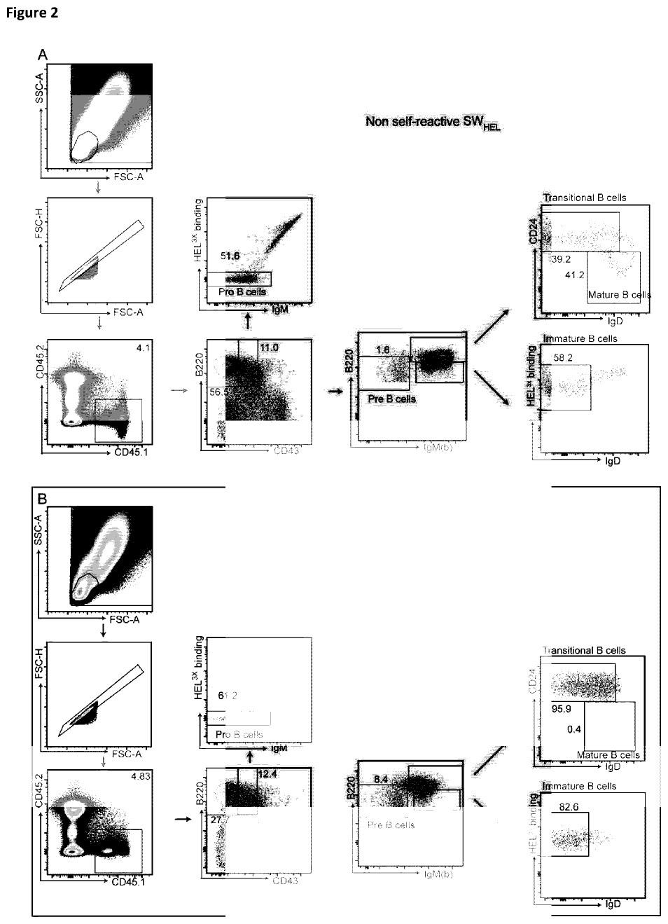

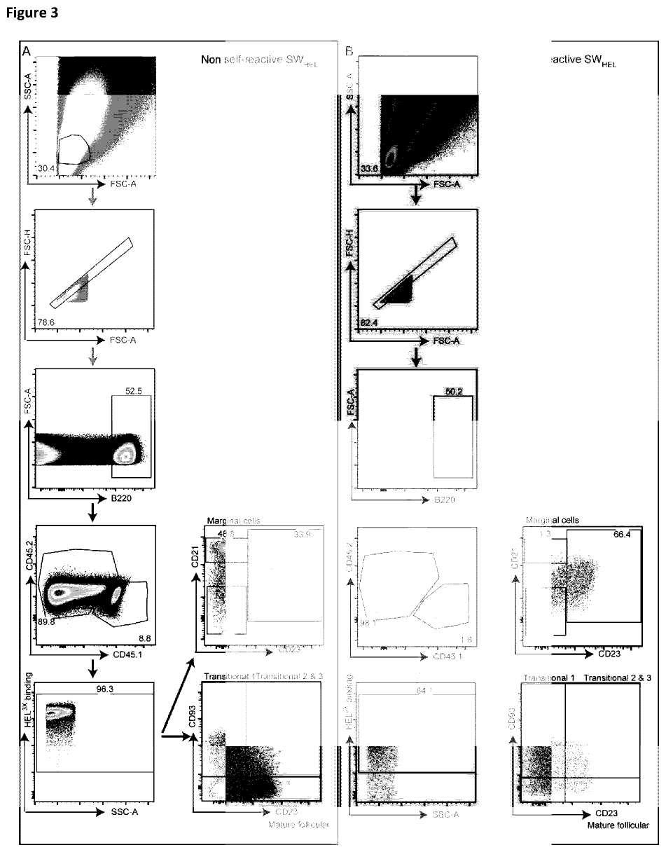

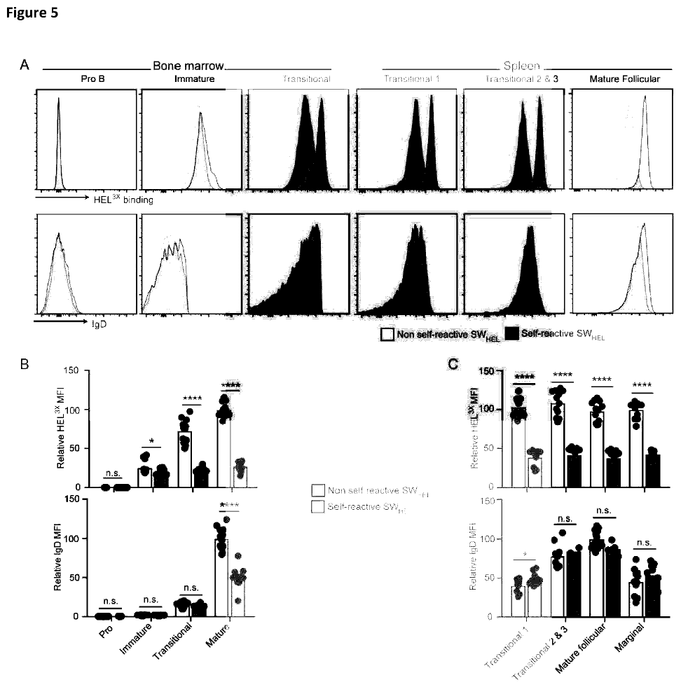

Bone marrow chimeric mice were engineered (FIG. 1A, B, FIGS. 2 to 5) as described in Example 2, in which the majority of developing B-cells reaching the spleen from the bone marrow are polyclonal and CD45.2⁺. However 1% of transitional B-cells and 0.1% of mature follicular B-cells were CD45.1⁺SW_{HEL} cells, which carry on their surface HyHEL10 antibody with a defined structure and low affinity for a self-protein (hen egg lysozyme with three substitutions (Phan et al. 2003; Phan et al. 2006; Padlan et al. 1989), HEL3X; 1/KD=1.2×10⁷ M−1) and for a structurally similar foreign protein (duck egg lysozyme, DEL; 1/KD=2.5×10⁷ M−1). In one group of chimeric mice, the self-protein was displayed on all cells as an integral membrane protein, mHEL3X, encoded by a transgene with a ubiquitin promoter. When SW_{HEL} B-cells were self-reactive, they reached the spleen as short-lived anergic cells with decreased surface IgM but normal surface IgD (FIG. 1B, FIGS. 2-5), located primarily in the T-cell zone (FIG. 1C). The frequency of anergic SW_{HEL} cells was lower than the circulating frequency of anergic IgD⁺ IgMlo VH4-34+B-cells that recognize ubiquitous cell surface antigens and mutate away from self-reactivity in humans.

It was firstly tested if self-reactive SW_{HEL} B-cells could respond to a foreign antigen that perfectly mimicked self. Sheep red blood cells (SRBCs) were covalently coupled with self-antigen at surface densities either the same as on the endogenous mouse red blood cells (MRBCs) or 30 fold higher (FIG. 1D). Despite equal T-cell help for germinal center (GC) responses by the diverse repertoire of other B-cells (FIG. 1F), self-reactive SW_{HEL} B-cells only entered GCs when SRBCs carried high antigen density (FIG. 1G). SRBCs with low antigen density could nevertheless induce GC responses from SW_{HEL} B-cells that were not self-reactive. These results are consistent with previous evidence that helper T-cells only cooperate with anergic B-cells when B-cell receptor cross-linking by foreign antigen is greater than that induced by self-antigen.

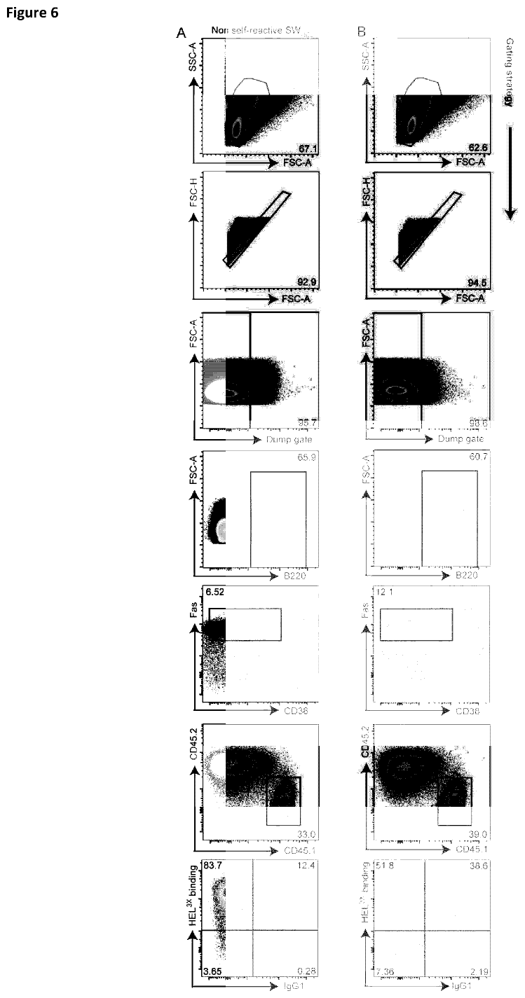

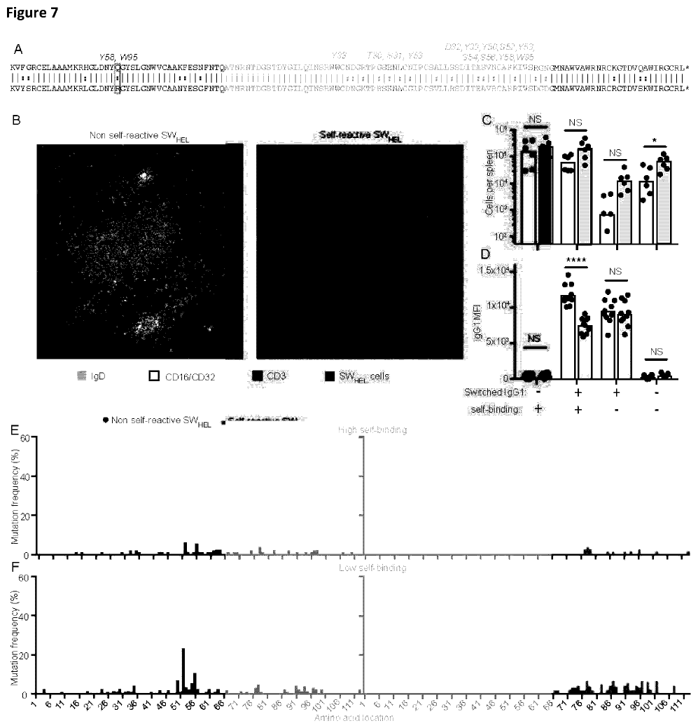

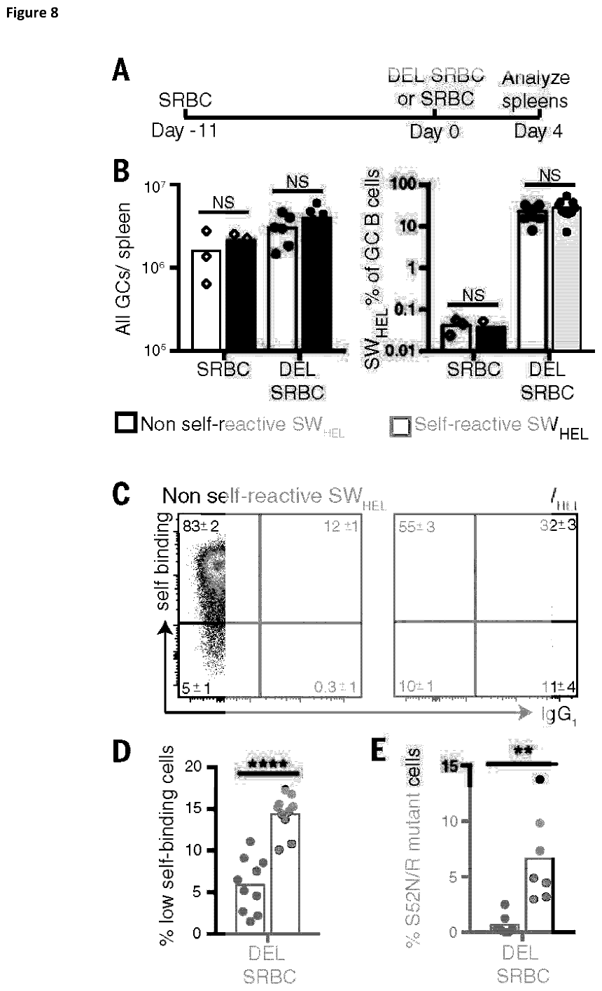

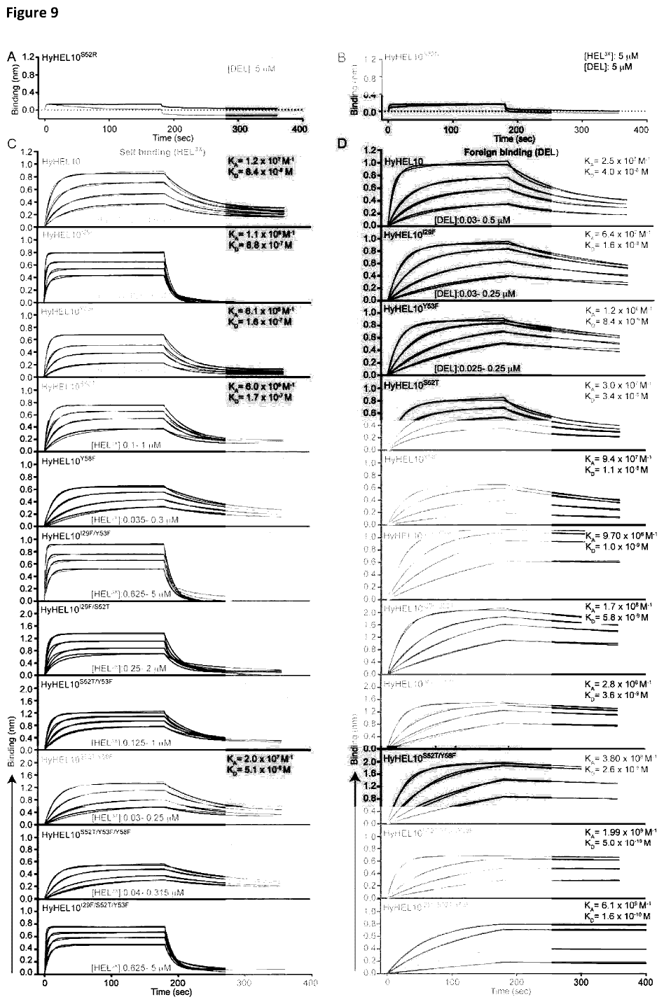

Next the response of self-reactive SW_{HEL} B-cells to DEL, which differs from self-antigen at four residues that contact the HyHEL10 H-chain (FIGS. 6 and 7A) was tested. GC reactions were initiated with unconjugated SRBCs and, 11 days later, SW_{HEL} B-cells recruited into the reaction synchronously by boosting with DEL coupled at high density to SRBCs (FIG. 8A). Four days after immunization with DEL-SRBCs, SW_{HEL} B-cells comprised ˜20% of all GC B-cells and were present in comparable total numbers regardless of self-reactivity (FIGS. 6, 7B and C and 8B). When the SW_{HEL} GC B-cells were self-reactive, they had lower surface IgG1 per cell (FIGS. 7D and 8C), likely caused by engagement with self-antigen on neighboring cells. At this early timepoint, the frequencies and numbers of IgG1⁻ and IgG1⁺SW^{HEL} B-cells with low binding to self-antigen were increased when the cells were self-reactive (FIGS. 7C and 8C and D). These low binding cells had increased frequencies of missense mutations (FIG. 7E, F), with 55% having acquired S52R or S52N mutations in complementarity determining region 2 (CDR2) (FIG. 8E). Both mutations drastically decreased affinity for both self and foreign protein (FIG. 9 and Table 1).

HEL^{3X} affinities (SELF)

| | | | | | | | | Expected |
|---|---|---|---|---|---|---|---|---|
| | | | | | | | ΔΔG | additive |
| | | | | | | Fold | apparent | ΔΔG |
| Mutant | K_{D} | k [symbol] | k [symbol] | K [symbol] | Affinity | change | (kcal/mol) | (k/cal/mol) |
| WT | 8.43 × 10 [symbol] | 1.49 × 10 [symbol] | 1.26 × 10 [symbol] | 1.19 × 10 [symbol] | 84 nM | 0.0 | 0.0 | |
| I29F | 8.81 × 10 [symbol] | 1.01 × 10 [symbol] | 8.89 × 10 [symbol] | 1.14 × 10 [symbol] | 880 nM | −10.5 | 1.4 | |
| S52T | 1.67 × 10 [symbol] | 1.21 × 10 [symbol] | 2.03 × 10 [symbol] | 5.98 × 10 [symbol] | 167 nM | −2.0 | 0.4 | |
| Y53F | 1.64 × 10 [symbol] | 1.21 × 10 [symbol] | 1.98 × 10 [symbol] | 6.11 × 10 [symbol] | 164 nM | −1.9 | 0.4 | |
| Y58F | 3.28 × 10 [symbol] | 2.16 × 10 [symbol] | 7.09 × 10 [symbol] | 3.05 × 10 [symbol] | 33 nM | 2.6 | −0.6 | |
| I29F/S52T | 6.10 × 10 [symbol] | 6.98 × 10 [symbol] | 4.26 × 10 [symbol] | 1.64 × 10 [symbol] | 610 nM | −7.2 | 1.2 | 1.8 |
| I29F/Y53F | 7.98 × 10 [symbol] | 9.85 × 10 [symbol] | 7.86 × 10 [symbol] | 1.25 × 10 [symbol] | 798 nM | −9.5 | 1.3 | 1.8 |
| S52T/Y58F | 5.14 × 10 [symbol] | 1.89 × 10 [symbol] | 9.69 × 10 [symbol] | 1.95 × 10 [symbol] | 51 nM | 1.6 | −0.3 | −0.2 |
| I29F/S52T/Y58F | 1.13 × 10 [symbol] | 1.14 × 10 [symbol] | 1.28 × 10 [symbol] | 8.88 × 10 [symbol] | 113 nM | −1.3 | 0.2 | 1.2 |
| S52T/Y53F | 1.52 × 10 [symbol] | 1.12 × 10 [symbol] | 1.71 × 10 [symbol] | 6.58 × 10 [symbol] | 152 nM | −1.8 | 0.3 | 0.8 |
| S52T/Y53F/Y58F | 2.44 × 10 [symbol] | 2.08 × 10 [symbol] | 5.07 × 10 [symbol] | 4.11 × 10 [symbol] | 24 nM | 3.5 | −0.7 | 0.2 |
| I29F/S52T/Y53F | 8.07 × 10 [symbol] | 8.64 × 10 [symbol] | 6.97 × 10 [symbol] | 1.24 × 10 [symbol] | 807 nM | −9.6 | 1.3 | 2.3 |

| | | | |
|---|---|---|---|
| S52R | Does not bind at 5 uM | N/A | N/A |
| S52R/Y53F | Does not bind at 5 uM | N/A | N/A |

DEL affinities (FOREIGN)

| | | | | | | | | Expected |
|---|---|---|---|---|---|---|---|---|
| | | | | | | | ΔΔG | additive |
| | | | | | | Fold | apparent | ΔΔG |
| Mutant | K_{D} | k [symbol] | k [symbol] | K [symbol] | Affinity | change | (kcal/mol) | (k/cal/mol) |
| WT | 4.01 × 10 [symbol] | 1.85 × 10 [symbol] | 2.40 × 10 [symbol] | 2.49 × 10 [symbol] | 40 nM | 0.0 | 0.0 | |
| I29F | 1.57 × 10 [symbol] | 1.76 × 10 [symbol] | 2.76 × 10 [symbol] | 6.37 × 10 [symbol] | 15 nM | 2.6 | −0.6 | |
| S52T | 3.35 × 10 [symbol] | 1.73 × 10 [symbol] | 3.79 × 10 [symbol] | 2.98 × 10 [symbol] | 33 nM | 1.2 | −0.1 | |
| Y53F | 8.35 × 10 [symbol] | 2.18 × 10 [symbol] | 1.82 × 10 [symbol] | 1.20 × 10 [symbol] | 8 nM | 4.8 | −0.9 | |
| Y58F | 1.07 × 10 [symbol] | 2.63 × 10 [symbol] | 2.81 × 10 [symbol] | 9.35 × 10 [symbol] | 11 nM | 3.8 | −0.8 | |
| I29F/S52T | 5.79 × 10 [symbol] | 1.55 × 10 [symbol] | 8.97 × 10 [symbol] | 1.73 × 10 [symbol] | 6 nM | 6.9 | −1.1 | −0.7 |
| I29F/Y53F | 1.03 × 10 [symbol] | 1.47 × 10 [symbol] | 1.52 × 10 [symbol] | 9.70 × 10 [symbol] | 1 nM | 39.0 | −2.2 | −1.5 |
| S52T/Y58F | 2.62 × 10 [symbol] | 1.74 × 10 [symbol] | 4.58 × 10 [symbol] | 3.80 × 10 [symbol] | 3 nM | 15.3 | −1.6 | −0.9 |
| I29F/S52T/Y58F | 1.69 × 10 [symbol] | 1.91 × 10 [symbol] | 3.22 × 10 [symbol] | 5.92 × 10 [symbol] | 2 nM | 23.8 | −1.9 | −1.5 |
| S52T/Y53F | 3.56 × 10 [symbol] | 2.00 × 10 [symbol] | 7.11 × 10 [symbol] | 2.81 × 10 [symbol] | 3.6 nM | 1.1 | −1.4 | −1.0 |
| S52T/Y53F/Y58F | 5.02 × 10 [symbol] | 2.54 × 10 [symbol] | 1.27 × 10 [symbol] | 1.99 × 10 [symbol] | 500 pM | 80.0 | −2.6 | −1.6 |
| I29F/S52T/Y53F | 1.64 × 10 [symbol] | 3.18 × 10 [symbol] | 5.22 × 10 [symbol] | 6.10 × 10 [symbol] | 164 pM | 245.0 | −3.3 | −1.6 |
| S52R | Does not bind at 5 uM | | | | N/A | N/A | | |
| S52R/Y53F | Does not bind at 5 uM | | | | N/A | N/A | | |
| | | | | | | Fold | | |
| I29F sisngle mutant binding to: | K_{D} | k [symbol] | k [symbol] | K [symbol] | Affinity | change | | |
| DEL (foreign) | 1.57 × 10 [symbol] | 1.76 × 10 [symbol] | 2.76 × 10 [symbol] | 6.37 × 10 [symbol] | 15 nM | 0.0 | | |
| HEL R21Q R73E D101K (self) | 8.81 × 10 [symbol] | 1.01 × 10 [symbol] | 8.89 × 10 [symbol] | 1.14 × 10 [symbol] | 880 nM | −56.1 | | |
| HEL R21Q R73E L75A D101R | 8.56 × 10 [symbol] | 9.34 × 10 [symbol] | 7.99 × 10 [symbol] | 1.17 × 10 [symbol] | 856 nM | −54.5 | | |
| (self with foreign 75) | | | | | | | | |
| HEL R21Q R73K D101R (self | 3.50 × 10 [symbol] | 1.92 × 10 [symbol] | 6.73 × 10 [symbol] | 2.86 × 10 [symbol] | 350 nM | −22.3 | | |
| with foreign 73) | | | | | | | | |
| HEL R21Q R73K L75A D101R | 2.77 × 10 [symbol] | 3.61 × 10 [symbol] | 1.00 × 10 [symbol] | 3.61 × 10 [symbol] | 28 nM | −1.8 | | |
| (self with foreign 73, 75) | | | | | | | | |
| HEL R21Q G71R R73K L75A | 3.18 × 10 [symbol] | 3.51 × 10 [symbol] | 1.12 × 10 [symbol] | 3.14 × 10 [symbol] | 32 nM | −2.0 | | |
| N77G D101R | | | | | | | | |
| (self with foreign 71, 73, 75, 77) | | | | | | | | |

[symbol]

indicates data missing or illegible when filed

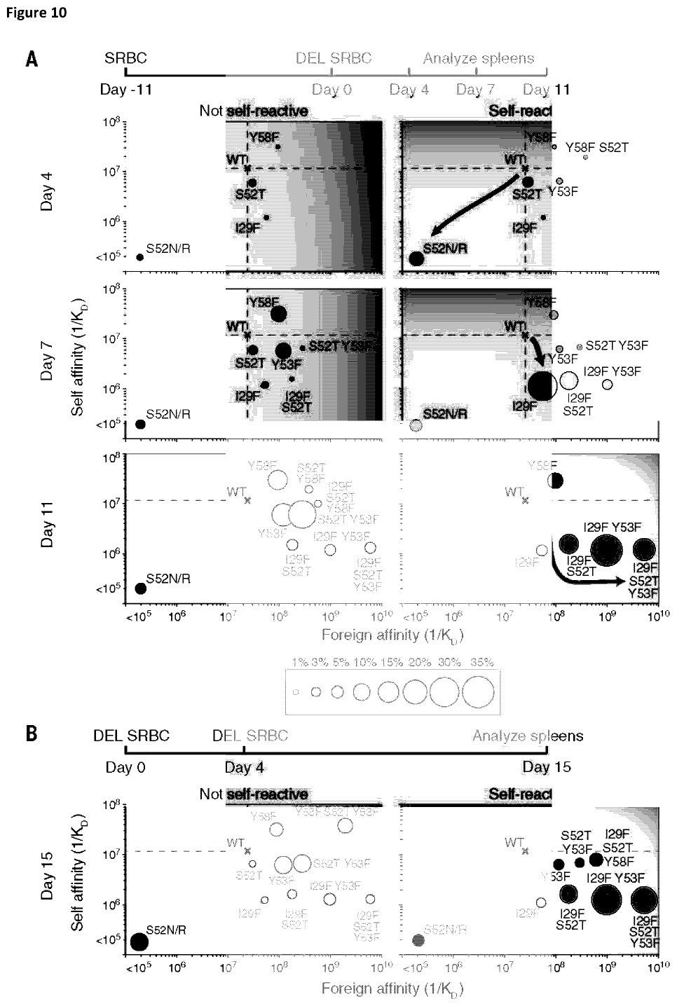

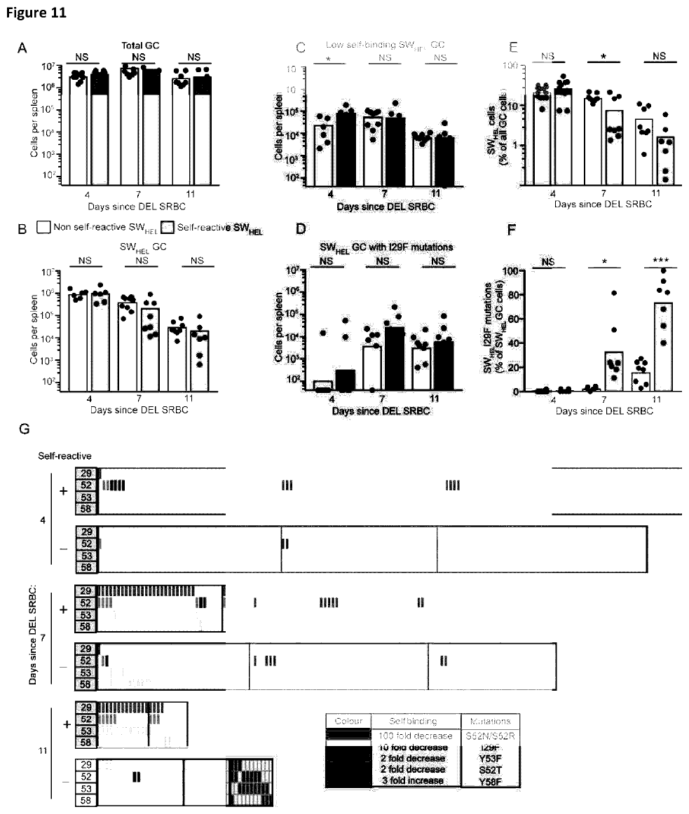

To determine if rapid selection for mutant GC B-cells with decreased affinity for self was followed by affinity maturation towards foreign, antibody mutations were analyzed 4, 7, and 11 days after SW_{HEL} B-cells were challenged with DEL-SRBC (FIGS. 10A and 11). On day 4, S52R or S52N mutations were again significantly increased when SW_{HEL} B-cells were self-reactive (11.55% vs 3.55%, P=0.0093). However, their frequency decreased on days 7 and 11. Instead, an I29F mutation in CDR1 became prevalent on day 7, occurring as a single substitution in 31% of SW_{HEL} B-cells when they were self-reactive compared with only 1.7% when they were not. I29F had the unique property of distinguishing foreign from self, causing a 10-fold decrease in self affinity and a 2.6-fold increase in foreign affinity (FIGS. 9, 10A, and Table 1).

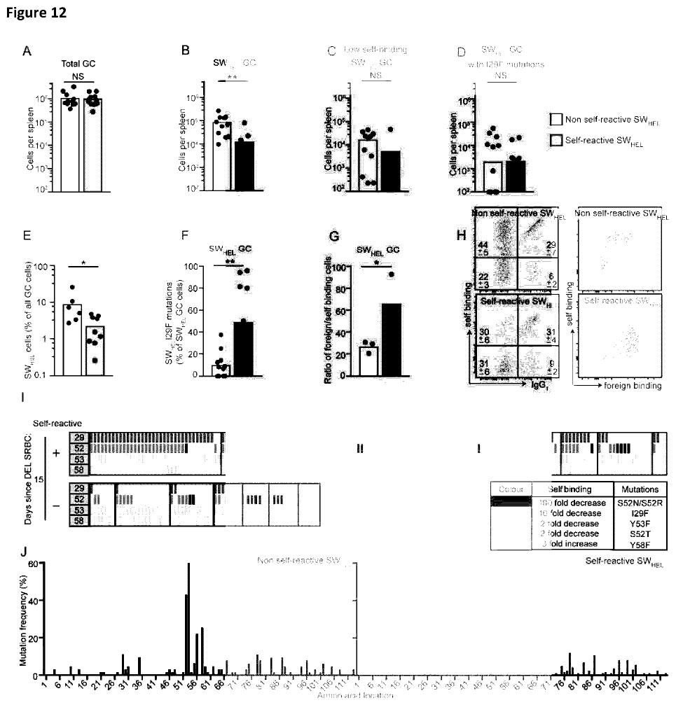

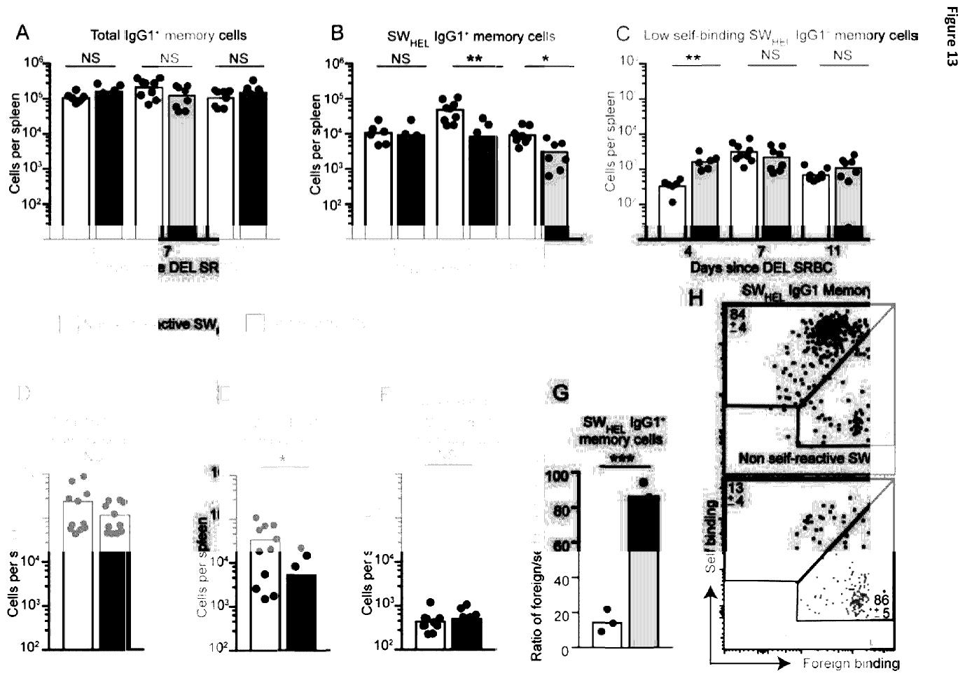

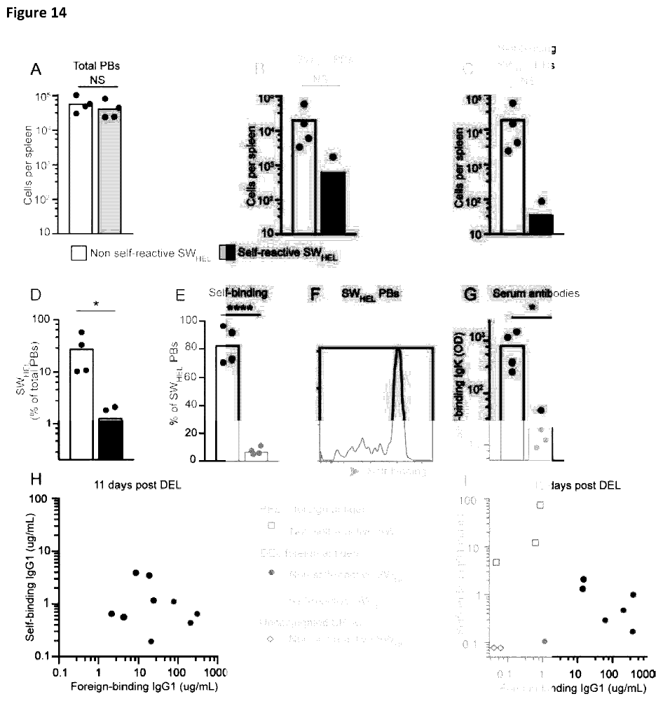

The I29F mutation became paired with S52T or Y53F mutations in CDR2, starting as a small subset of self-reactive cells on day 7 but becoming the most prevalent as pairs or a trio by day 11. S52T or Y53F were rarely found individually but combined with the I29F foundation mutation increased foreign-self discrimination, retaining 1×10⁶ M⁻¹ affinity for self but progressively increasing foreign affinity to 6×10⁹ M⁻¹. Strong epistatic (non-additive) effects were observed. For example, the I29F S52T YF3F trio increased the apparent AAG for binding foreign antigen by −3.3 kcal/mol, compared with −1.6 kcal/mol expected for additive effects of the individual mutations (Table 1). This trio of mutations became even more prevalent when self-reactive SW_{HEL} B-cells were recruited at the outset of the GC reaction and analyzed 15 days later (FIGS. 10B and 12). Thus, an antibody that was initially unable to distinguish foreign from self had evolved 5000-fold differential binding to foreign over self, by first mutating away from binding self and then towards binding foreign. SW_{HEL}-derived cells that had lost self-binding but retained foreign were also frequent among the IgG1⁺ memory B-cell compartment (FIG. 13). Foreign-specific IgG1 serum titers were increased in mice with initially self-reactive SW_{HEL} B-cells (FIG. 14).

A different, less optimal evolutionary trajectory prevailed when SW_{HEL} B-cells were not self-reactive, dominated by acquisition of a CDR2 mutation, Y58F, either alone, paired, or in trio with S52T and Y53F (FIG. 10). Y58F alone or with S52T and Y53F increased self-affinity fourfold, explaining why this trajectory was not taken by self-reactive SW_{HEL} cells. The Y58F-S52TY53F trio increased foreign affinity to 2×10⁹ M−1, which was three-fold lower than the I29FS52T-Y53F trio selected through the self-reactive trajectory.

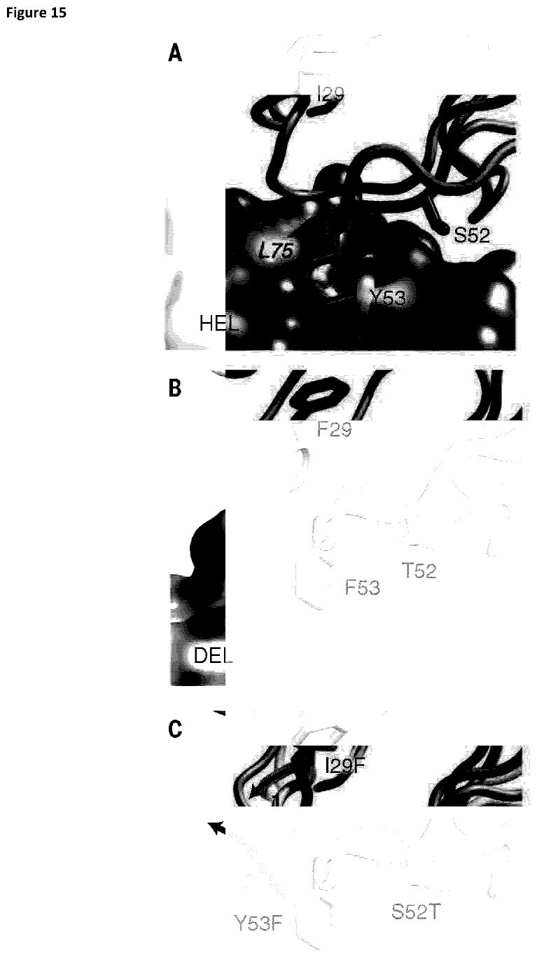

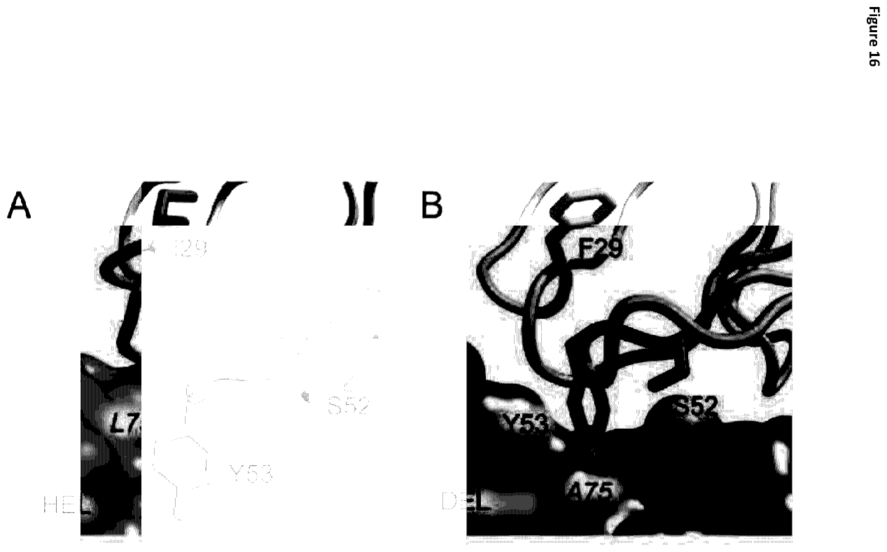

To understand how these three mutations conferred a 5000-fold differential binding to foreign over self, we used X-ray crystallography to analyze the structure of HyHEL10^{I29F, S52T, Y53F} complexed with DEL (FIG. 15 and Table 2) compared to HyHEL10^{WT} complexed with HEL. I29F resulted in a structural rearrangement of the CDR1 loop to accommodate the larger phenylalanine side chain. Displacement of this loop opened up additional structural adjustments of CDR2, and in particular repositioned Y53F to interact with a hydrophobic pocket formed on the surface of DEL by the short Alanine 75 side chain compared to the much longer Leucine in HEL. The CDR2 backbone adjustments also allowed replacement of the smaller S52 side chain with threonine. Thus, the structural analyses were in agreement with the observed mutational trajectory, whereby the I29F foundation mutation introduces structural rearrangements into CDR1 and CDR2; these enable secondary mutations at positions 52 and 53, which selectively increase foreign affinity in an epistatic manner. Binding studies confirmed I29F confers 50-fold lower binding to self versus foreign antigen by exploiting the L75A foreign pocket coupled with the adjacent E73K charge reversal (Table 1). This was further confirmed by solving the structure of HyHEL10^{I29F} Complexed with DEL (FIG. 16).

TABLE 2

| | | |
|---|---|---|
| | HyHEL10- | HyHEL10-I29F-S52T- |
| Crystal | I29F:DEL-I | Y53F:DEL-I |

Data collection statistics

| | | |
|---|---|---|
| Wavelength | 0.9537 | 0.9537 |
| Spacegroup | P 2₁ 2 2₁ | P 2₁ 2₁ 2₁ |
| Unit cell dimensions: | 89.02, 102.91, 133.98; | 88.65, 132.66, 199.11; |
| a, b, c (Å); α, β, γ, (°) | 90.0, 90.0, 90.0 | 90.0, 90.0, 90.0 |
| Resolution range | 2.43-47.49 (2.43-2.51) | 1.90-49.78 (1.90-1.93) |
| Total reflections | 459,563 (43,523) | 1,484,865 (69,377) |
| Unique reflections | 47,296 (4,593) | 180,881 (8,562) |
| Completeness | 99.9 (100) | 98.0 (94.6) |
| Multiplicity | 9.7 (9.5) | 8.2 (8.1) |
| Average (I/σ(I)) | 15.5 (3.3) | 12.6 (2.8) |
| Mean half set correlation, | 0.998 (0.850) | 0.998 (0.642) |
| CC_{1/2} | | |
| Rmeas (all I+ and I−) | 0.100 (0.894) | 0.105 (0.838) |
| Rpim all (I+ and I−) | 0.032 (0.261) | 0.035 (0.286) |
| Wilson B (Å²) | 46.1 | 23.5 |

Refinement and model statistics

| | | |
|---|---|---|
| Reflections used | 44,719 (3,245) | 171,765 (12,115) |
| R_{work}/R_{free} | 0.262/0.296 | 0.193/0.235 |
| Fab-DEL complexes/asu | 2 | 4 |
| Atoms protein | 8176 | 16869 |
| B average protein (Å²) | 61.9 | 29.5 |
| Atoms non-protein | 50 | 1034 |
| B average non-protein | 41.7 | 30.6 |
| (Å²) | | |
| RMSD bond lengths (Å²) | 0.0108 | 0.0119 |
| RMSD bond angles (°) | 1.44 | 1.54 |
| Ramachandran Outliers | 0.37 | 0.05 |
| (%) | | |
| Ramachandran Favored | 97.2 | 98.1 |
| (%) | | |
| PDB entry | 5VJO | 5VJQ |

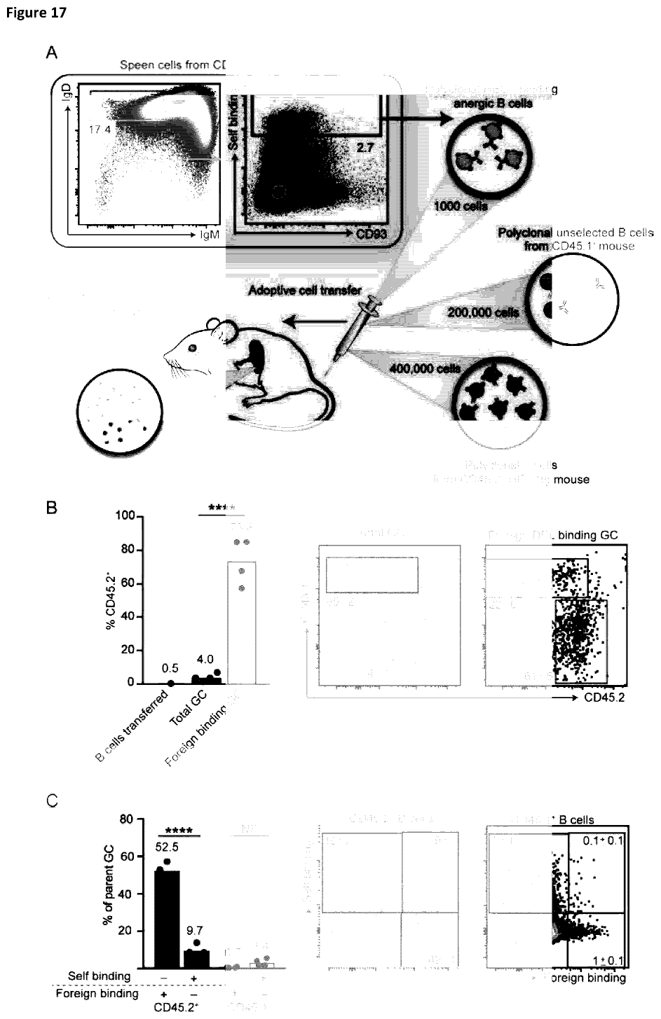

Anergic B-cells in the mHEL3×tg mice were next identified within a polyclonal repertoire that possessed micromolar affinity for the same self antigen and tested if they too could resolve antigenic mimicry. HEL^{3X}-binding B-cells comprised 2.7% of IgD⁺IgMl® anergic B-cells and 0.5% of all spleen B-cells (FIG. 17A). These were sorted and added at 0.5% frequency to unselected CD45.1⁺ B-cells, and the polyclonal mixture injected with T-cells into mHEL^{3X}tg Rag1^{-/-} mice immunized with DEL-SRBC. In the recipients, 96% of the GC response was derived from the unselected CD45.1⁺ B-cells, presumably mostly recognizing SRBC antigens. By contrast, 61% of the DEL-binding GC response was derived from the polyclonal HEL^{3X}-binding anergic CD45.2⁺ B-cells (FIG. 17B). Only 9.7% of these cells still bound self-antigen, whereas 53% bound foreign DEL selectively (FIG. 17C). Thus, in a normal repertoire, cells with micromolar affinity for self-HEL^{3X} are dominant contributors to the GC response against the self-mimic DEL and rapidly lose binding to self.

The findings here provide evidence for autoantibody redemption in human antibodies by showing mutation away from self precedes mutation towards foreign to create unique epistatic trajectories. Self-reactivity, rather than being a barrier to immunization, directed cells down an alternative trajectory, which produced a higher final affinity against the foreign immunogen.

### Example 17. Vaccination with a Conformationally Flexible Antigen

The affinity maturation pathways of an antibody that cross-reacts with a conformationally flexible protein antigen as well as a self-antigen were investigated. Specifically, the role of epitope flexibility in both non self-reactive and self-reactive systems were explored concurrently.

To further investigate the role of self-reactivity constraints on broadly neutralizing antibody production bone marrow chimeric mice in which the majority of mature B-cells were polyclonal and CD45.2⁺ were engineered in accordance with Example 2. However, 0.1% of mature follicular B-cells expressed the HyHEL10, antibody as described previously in Example 16. In one group of chimeric mice, mHEL^{3X}, a defined low affinity self protein was displayed on all cells as an integral membrane protein (Chan, et al. 2012). As described above in Example 16, when the mice were immunized with a similar affinity protein, structurally similar to the self-antigen (duck egg lysozyme, DEL) self-reactive HyHEL10 B cells are able to acquire mutations that specifically lowered their affinity to the self-antigen while maintaining foreign binding. Thus to further this phenomenon the structure of HEL^{3X} was solved and compared directly to DEL. This revealed the backbone of the self and foreign antigens to be remarkably similar with the main differences relating largely to surface residues, particularly around location 73.

As described above in Example 16, mice were immunized with sheep red blood cells (SRBC) conjugated to the foreign antigen identical (HEL^{3X}) or slightly differing from self (DEL), both with a near identical affinity to HyHEL10 (1/K_{D}=1.2×10⁷ M⁻¹ vs 2.5×10⁷ M⁻¹) to investigate the importance of similarity between the self and foreign antigen. Although the overall GC and memory response was comparable with these two different antigens when the self-antigen and the foreign antigen were identical HyHEL10 B cells were significantly less frequent in the GC and memory compartment Analysis with the serum IgG₁ compartment revealed that mice with self-HEL^{3X} immunized with foreign DEL were actually able to generate 26 fold higher IgG₁ antibody titers than those without self-mHEL^{3X}, as was shown in Example 16. In contrast, although the mice immunized with foreign HEL^{3X} were able to form 70 fold higher HEL^{3X} IgG₁ antibody titers than those mice immunized with unconjugated SRBC, it remained 10 fold lower than that achieved in the non self-reactive mice.

As seen previously in Example 16, when mice with self-mHEL^{3X} were immunized with foreign DEL, this rapidly led for the acquisition of a mutational trajectory which resulted in the formation of clones that had dramatically decreased affinity for the self-antigen while concurrently increasing foreign binding affinity. When the control non-tg mice were immunized with foreign HEL^{3X}, where the HyHEL10 cells responded in the absence of self-mHEL^{3X}, high frequencies of HyHEL10 GC cells accumulated with Y53D (84% of cells), Y58F (10%) or both (5%. Y53D increases HEL^{3X} affinity 100-fold (Phan, et al. 2006, Chan, et al. 2012). By contrast Y53D was virtually absent among HyHEL10 GC cells in HEL³tg mice where they were exposed to foreign and self-HEL^{3X}. In the presence of self-mHEL^{3X}, 61% of HyHEL10 GC cells elicited by of HEL^{3X}-SRBC carried S52R, including 37% where S52R was paired with Y53F. Bio-layer interferometry revealed that the S52R mutation, alone or in combination with the Y53F mutation, completely abolished measurable binding to the highest concentration of HEL^{3X} tested, representing at least 100-fold lower affinity. Thus when foreign erythrocytes carried a with identical amino acid sequence to a protein on self-erythrocytes, despite reactivation of anergic B cells into the germinal center subsequent affinity maturation of HyHEL10 B cells in GC was completely suppressed. Instead the cells were selected for loss of affinity. This is similar to the what has been seen previously when HEL was the self and foreign antigen whereby HyHEL10 GC B cells inserted a glycosylation to decrease, but not entirely remove, self and foreign binding (Sabouri, et al. 2014), however in this scenario, where the B cell affinity to the self and foreign antigen began at a much lower, physiological affinity, these mutations completely abolished binding to both antigens.

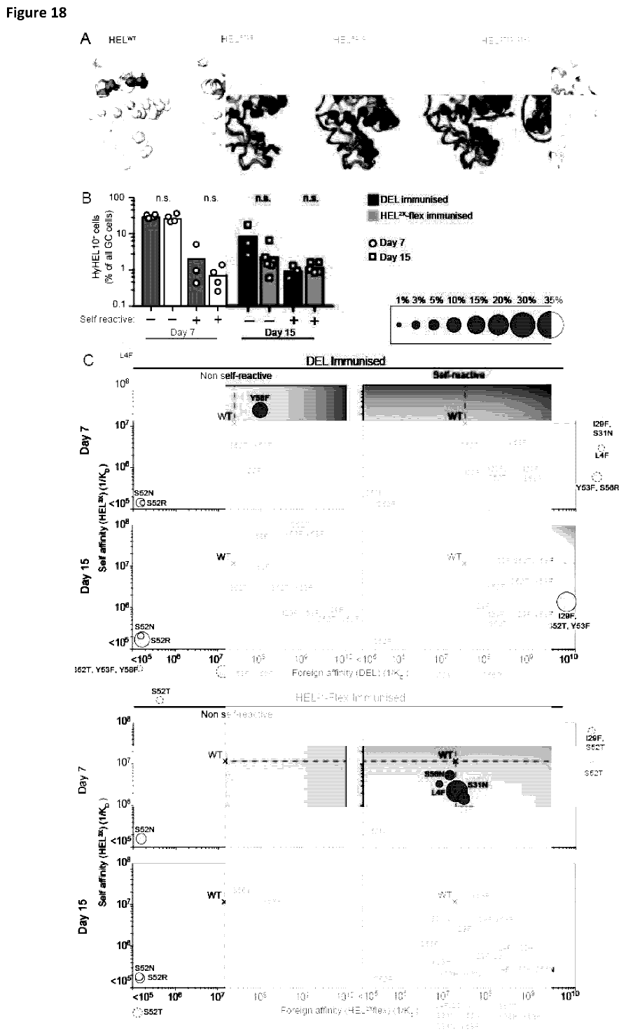

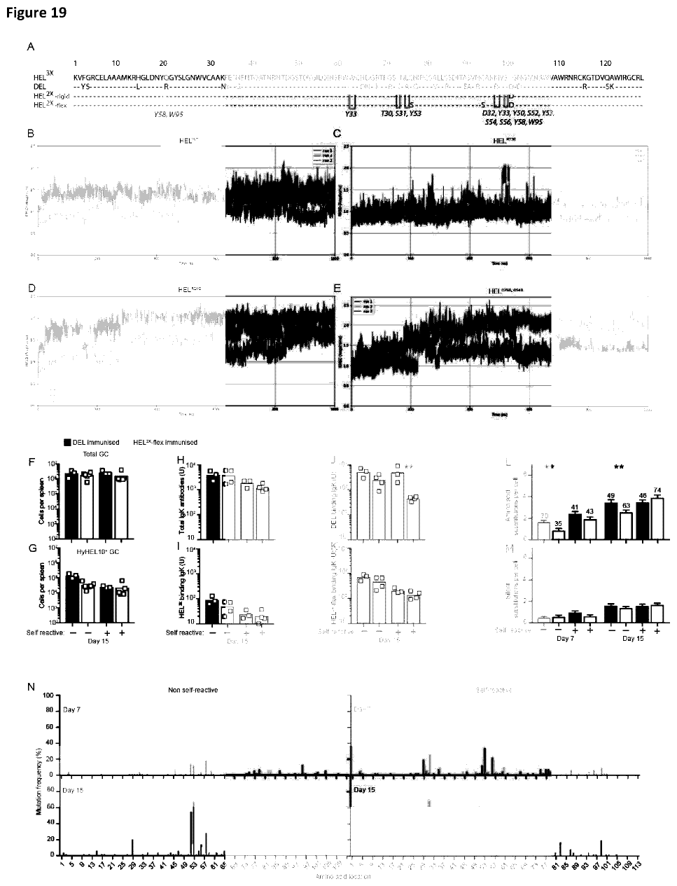

HEL is characterized to have four disulphide bonds all of which contribute to the structural stability of the molecule (Inaka, et al. 1991, Buck, et al. 1995, Yokota, et al. 2000). To investigate the role of epitope flexibility in neutralizing antibody responses a variant of HEL^{R21Q,R73E,C76S,C94S} (HEL^{2X}-flex) lacking one of these disulphide bonds (FIG. 19A) was generated. This resulted in local unfolding around the removal of the disulfide bond, which stabilizes the native fold as confirmed by MD simulations (FIG. 18A). The increased flexibility around the C94S site appears to propagate along the helix, allowing increased fluctuation of residues 101-104, which are important for antigen binding. At the other, C76S end of the disulfide bond, the region containing HyHEL10-epitope residues R73 and L75 becomes looser. The behavior of HEL C76S/C94S epitope in vivo is thus predicted to be due to increased flexibility of important binding-related residues.

To determine the effect of epitope flexibility, the responses of HEL^{2X}-flex to DEL, with both antigens being extremely similar in regards to HyHEL10 affinity (1/K_{D}=1.6×107 M⁻¹ for HEL^{2X}-flex vs 1/K_{D}=2.5×107 M⁻¹ for DEL) and having comparable contact residue changes from the self-antigen (FIG. 19A) were compared. Immunization HEL^{2X}-flex-SRBC resulted in similar recruitment and maintenance of HyHEL10 B cells in the GC as DEL immunization in a self-reactive and non self-reactive system (FIGS. 18B and 19F and G). Overall, HyHEL10 cells from mice immunized with HEL^{2X}-flex or DEL were similarly able to both avoid self-reactivity and neutralize their respective foreign antigen, in regards to both affinity specific mutations and total serum antibody titers. In self-reactive cells, similar to what is seen for DEL immunization (Burnett et al. 2018), initially we saw a mutation away from self-reactivity followed by mutations towards the foreign antigen (FIGS. 18 C and D, 20). Similar to what was seen in DEL immunization, but contrary to what we found when the self and foreign antigen were identical in the HEL^{3X} immunization, immunization of self-reactive HyHEL10 B cells with the flexible epitope resulted in the development of clones with higher foreign binding than their non self-reactive counterparts (FIGS. 18C and D, 20). However in the case of DEL immunization cells were able to achieve foreign neutralizing affinity of greater than 1/K_{D}=108M⁻¹, while concurrently avoiding self-antigen binding, with the addition of two amino acid mutations, a concurrently foreign neutralizing ability required three to four somatic mutations.

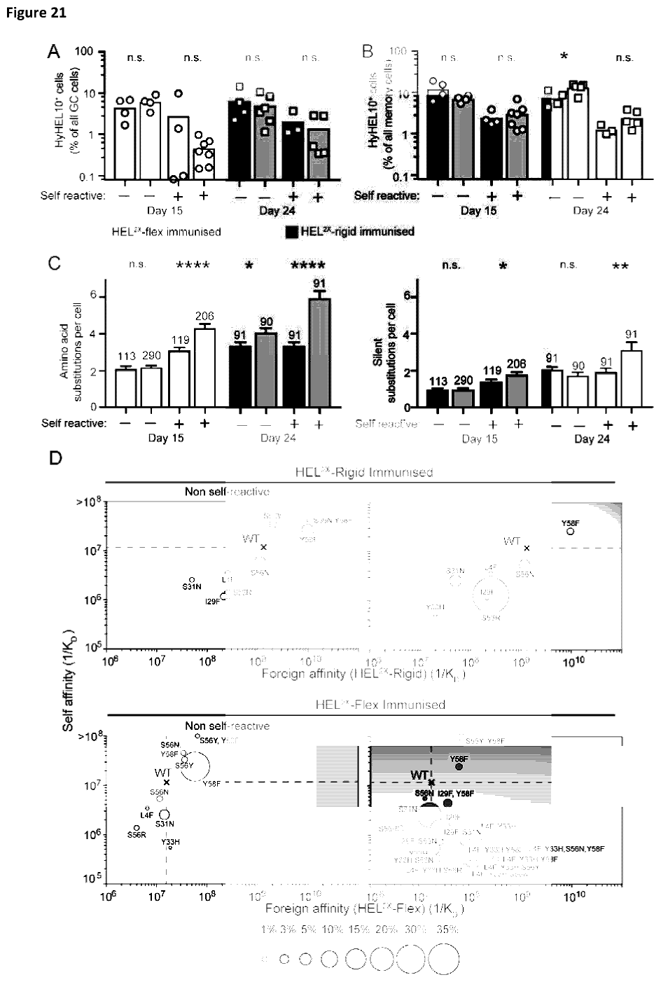

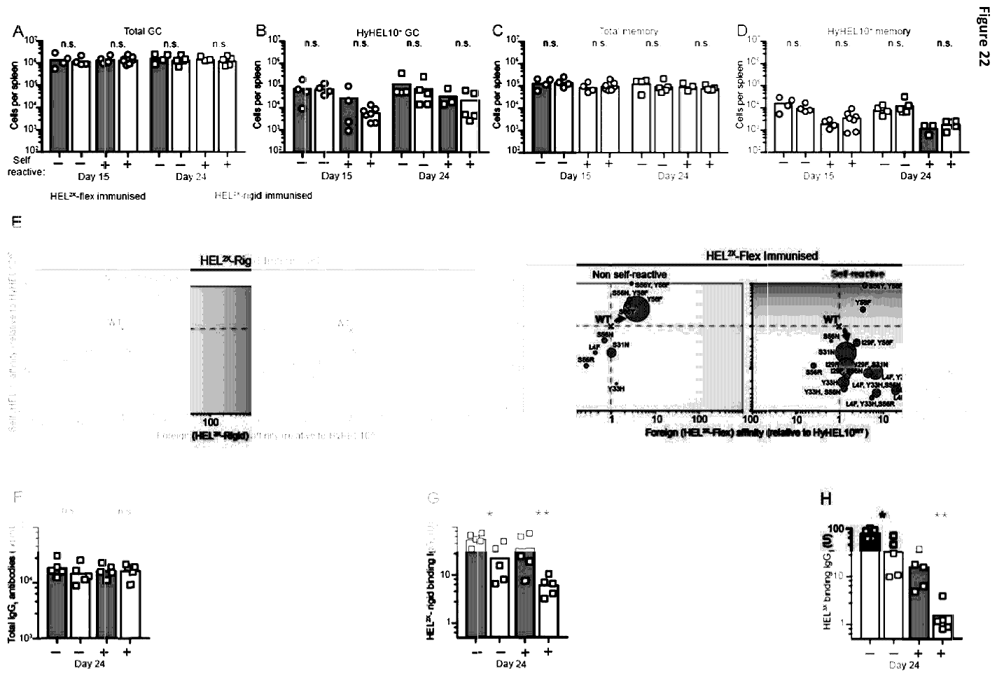

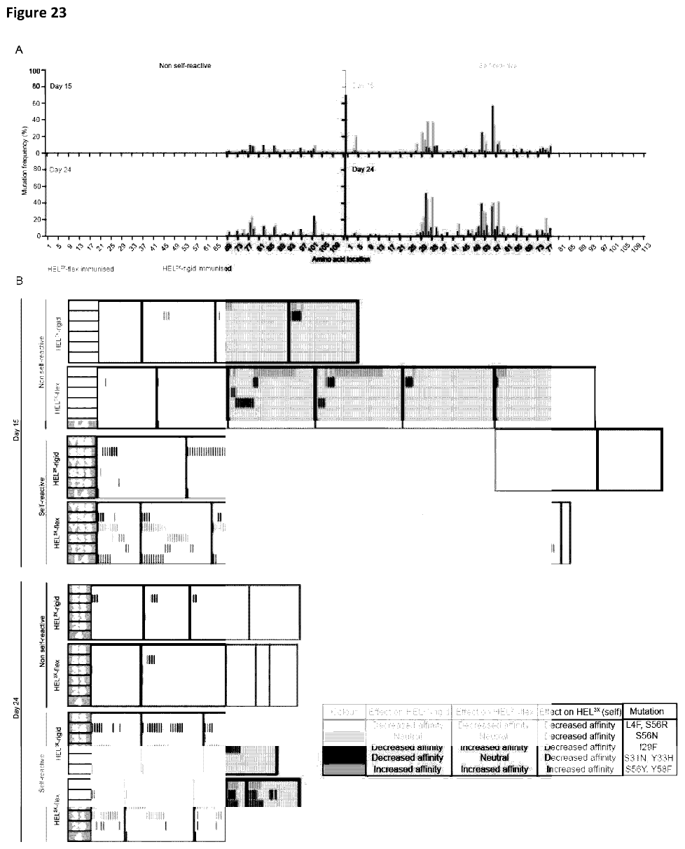

Although HEL^{2X}-flex and DEL have near identical affinity for HyHEL10 direct comparison between them is somewhat confounded by the structural differences between the two proteins. To precisely determine the effect of epitope flexibility on neutralizing antibody production, a variant of HEL HEL^{R21Q,R73E} (HEL^{2X}-rigid) that had the same contact residue mutations as HEL^{2X}-flex but maintained the disulfide bond, giving it a more rigid conformational structure (FIGS. 18A and 19A) was generated. Immunization with either of these proteins revealed that self-reactive and non self-reactive B cells were able to recognize the flexible epitope as evidenced by their equal recruitment of HyHEL10 B cells into the GC compartment and normal, if not slightly increased, recruitment into the memory compartment (FIGS. 21A and B and 22A-D). When their somatic hypermutation profile was examined, the flexible epitope was found to result in higher mutation load, particularly evident in when the antigen was self-reactive (FIG. 21C). Overall, HyHEL10 B cells were effectively able to generate mutations with neutralizing affinity to both the rigid and flexible epitope (FIGS. 21D and 23). However again it was found that in the self-reactive system for B cells to achieve foreign neutralizing affinity of greater than 1/K_{D}=10⁸ M⁻¹, they required three to four somatic hypermutations, whereas in the rigid antigen scenario a single mutation was sufficient to concurrently lower self-affinity while maintaining foreign neutralizing capacity (FIGS. 21D and 22E). Although a higher mutational load was required for the flexible antigen to maintain foreign neutralizing affinity while avoiding self reactivity, it is interesting to note that when HyHEL10 B cells did achieve sufficient mutational burden to neutralize the foreign antigen they were actually more effective at mutating away from self than the rigid antigen counterpart. In the case of the flexible epitope, 33.9% of these clones had lowered their affinity below 1/K_{D}=10⁶M⁻¹, which only occurred in 0.8% of clones from mice immunized with the rigid epitope (FIG. 21D). Similar, self-reactive mice immunized with the flexible epitope had 10 fold lower HEL^{3X} binding IgG₁ than those immunized with the rigid epitope (FIG. 22H).

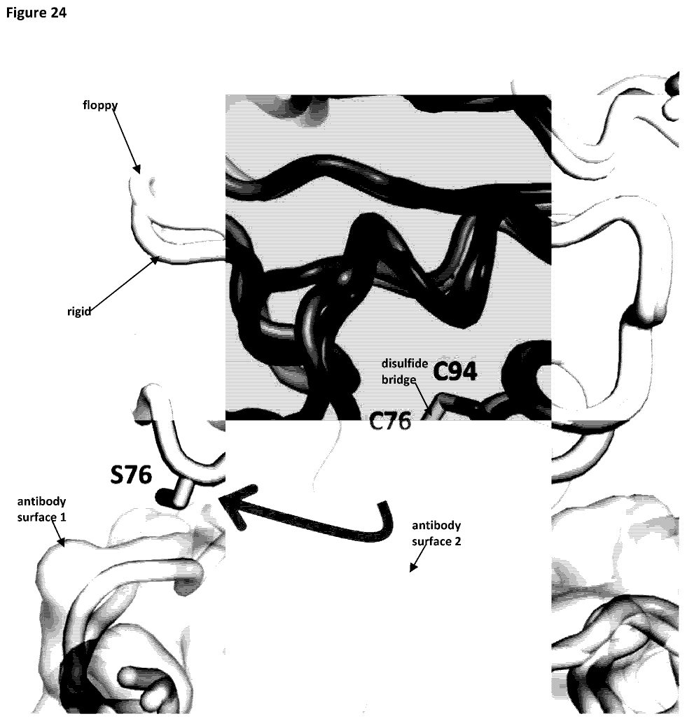

Given the dramatic differences in the mutational hierarchy achieved by HyHEL10 neutralizing the rigid or flexible variant of the epitope, the crystals of several HyHEL10 complexes were explored to further understand this variation. Crystals of the highest affinity neutralizing clone enriched in the self-reactive HyHEL10 GC B cells, HyHEL10^{L4F, Y33H, S56N, Y58F}, in combination with HEL^{2X}-flex were generated to further elucidate how the antibody was able to neutralize the flexible epitope of HEL^{2X}-flex while avoiding binding to the self-antigen. This was compared to the structure of the HyHEL10^{L4F, Y33H, S56N, Y58F} antibody alone. A complex of the HyHEL10 antibody complexed with HEL^{2X}-rigid, which has similar foreign binding affinity as the HyHEL10^{L4F, Y33H, 56N, Y58F} HEL^{2X}-flex complex was also generated as a control. Despite these two antibodies having similar foreign binding affinities, the flexible nature of the disulfide bond meant that the antibody antigen complex behaved in a drastically different manner in the two situations (FIG. 24).

The findings here extend the evidence that autoantibody redemption could be utilized as a potential strategy to generate bNAs for challenging antigens, including those utilizing epitope flexibility. GC B cells are able to concurrently overcome the challenges imposed by molecular mimicry and epitope flexibility to generate high affinity neutralizing antibodies. However a high mutational load including non-canonical framework region mutations are required to overcome these challenges concurrently. This suggests that potentially the limitations currently preventing bNA production for important pathogen neutralization may in fact be probabilistic, rather than biophysical or thermodynamic. This supports previous evidence suggesting that a high frequency of germline precursors may be a limiting factor in HIV bNA production (Abbott, et al. 2018) and may help to explain the highly mutated and atypical nature of HIV bNAs and explain why they are only generated in a limited number of patients (Klein, et al. 2013, West, et al. 2014).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### REFERENCES

Abbott et al. (2018) Immunity 48(1): 133-146 e136.
Al-Lazikani et al. (1997) J Mol Biol 273: 927-948.
Buck et al. (1995) Biochemistry 34: 4041-4055.
Burnett et al. (2018) Science 360: 223-226.
Chan et al. (2012) Immunity 37, 893-904.
Chothia CAM and Lesk J (1987) Mol Biol 196:901-917.
Chothia et al. (1989) Nature 342:877-883.
De Genst et al. (2006) Proc. Natl. Acad. Sci. U.S.A. 103:4586-4591.
Inaka et al. (1991) J Biol Chem 266: 12599-12603.
Klein et al. (2013) Cell 153(1): 126-138.
Padlan et al. (1989) Proc. Natl. Acad. Sci. U.S.A. 86: 5938-5942.
Phan et al. (2003) J. Exp. Med. 197: 845-860.
Phan et al. (2006) J. Exp. Med. 203: 2419-2424.
Paus et al. (2006) J. Exp. Med. 203: 1081-1091.
Sabouri et al. (2014) Proc Natl Acad Sci 111: 2567-2575.
West et al. (2014) Cell 156: 633-648.
Yokota et al. (2000) J. Mol Biol 295: 1275-1288.

1. A method for generating an antibody having improved specificity and/or affinity for a target antigen, the method comprising:
i) introducing the target antigen in an animal, wherein the animal expresses a variant antigen that is structurally related to the target antigen, and the animal further comprises B-cells encoding an antibody with a basic specificity and/or affinity for the target antigen, whereby the B-cells express a B-cell receptor comprising the antibody;
ii) allowing the B-cells to undergo affinity maturation in the animal; and
iii) isolating B-cells that have undergone affinity maturation in the animal;
whereby the B-cells isolated in iii) express antibodies with improved specificity and/or affinity for the target antigen compared to antibodies expressed by the B-cells prior to affinity maturation.

2. A method for generating an antibody having improved specificity and/or affinity for a target antigen, the method comprising:
i) introducing into an animal a B-cell, the B-cell encoding an antibody with a basic specificity and/or affinity for the target antigen, whereby the B-cell expresses a B-cell receptor comprising the antibody, and wherein the animal expresses a variant antigen that is structurally related to the target antigen;
ii) introducing the target antigen in the animal;
iii) allowing the B-cells to undergo affinity maturation in the animal; and
iv) isolating the B-cells that have undergone affinity maturation in the animal;
whereby the B-cells isolated in iv) express antibodies with improved specificity and/or affinity for the target antigen compared to antibodies expressed by the B-cells prior to affinity maturation.

3. The method of claim 1 or claim 2, wherein the B-cells have been genetically modified to encode the antibody with the basic specificity and/or affinity for the target antigen.

4. The method of claim 2 or claim 3, wherein introducing the B-cell into the animal comprises irradiating the animal and transplanting the B-cells into the animal.

5. The method of any of claims 1 to 4, wherein the basic affinity of the antibody for the target antigen is low affinity relative to a desired improved affinity.

6. The method of claim 5, wherein the low affinity for the target antigen is a K_{D} of about 10⁻⁶ M to about 10⁻⁸ M.

7. The method of any of claims 1 to 6, wherein the improved affinity is an increase in affinity for the target antigen of at least 100-fold.

8. The method of claim 7, wherein the improved affinity is an increase in affinity for the target antigen of at least 1000-fold.

9. The method of any of claims 1 to 8, wherein the improved specificity for the target antigen is an at least 10-fold increase in specificity.

10. The method of claim 9, wherein the improved specificity for the target antigen is an at least 100-fold increase in specificity.

11. The method of claim 9, wherein the improved specificity for the target antigen is an at least 1000-fold increase in specificity.

12. The method of any of claims 1 to 11, wherein the target antigen is a peptide or polypeptide antigen.

13. The method of claim 12, wherein the variant antigen comprises at least one variant amino acid compared to the target antigen.

14. The method claim 13, wherein the variant antigen comprises at least one variant amino acid residue in an epitope to which the antibody binds.

15. The method of claim 14, wherein the variant antigen comprises at least one variant amino acid residue in a surface that contacts the antibody heavy chain.

16. The method of any one of claims 1 to 15, wherein the variant antigen is encoded by a transgene in the animal.

17. The method of claim 16, wherein the transgene comprises a ubiquitin promoter for expression of the variant antigen.

18. The method of any one of claims 1 to 11, wherein the target antigen is a carbohydrate antigen or hapten.

19. The method of claim 18, wherein the carbohydrate antigen is selected from a glycoprotein, glycolipid, polysaccharide and glycoconjugate antigen.

20. The method of claim 18 or claim 19, wherein the carbohydrate antigen is a cancer cell antigen.

21. The method of claim 18 or claim 19, wherein the target antigen is a cancer neo-antigen.

22. The method of claim 18 or claim 19, wherein the carbohydrate antigen is a bacterial or viral carbohydrate antigen.

23. The method of any of claims 1 to 22, wherein the animal is a mouse or rat.

24. A method for generating an antibody having improved specificity and/or affinity for a target antigen, the method comprising:
i) genetically modifying a B-cell in an animal to encode an antibody with a basic specificity and/or affinity for the target antigen, whereby the B-cell expresses a B-cell receptor comprising the antibody, and wherein the animal expresses or is inoculated with the target antigen and expresses a variant antigen that is structurally related to the target antigen,
ii) allowing the B-cells to undergo affinity maturation in the animal; and
iii) isolating B-cells that have undergone affinity maturation in the animal;
whereby the B-cells isolated in iii) express antibodies with improved specificity and/or affinity for the target antigen compared to antibodies expressed by the B-cells prior to affinity maturation.

25. A method for generating an antibody having improved specificity and/or affinity for a target antigen, the method comprising:
i) genetically modifying a B-cell to encode an antibody with a basic specificity and/or affinity for the target antigen, whereby the B-cell expresses a B-cell receptor comprising the antibody;
ii) introducing the genetically modified B-cell in i) into an animal expressing or inoculated with the target antigen and expressing a variant antigen that is structurally related to the target antigen;
iii) allowing the B-cells to undergo affinity maturation in the animal; and
iv) isolating B-cells that have undergone affinity maturation in the animal;
whereby the B-cells isolated in iv) express antibodies with improved specificity and/or affinity for the target antigen compared to antibodies expressed by the B-cells prior to affinity maturation.

26. A method for providing an antibody having improved specificity and/or affinity for a target antigen, the method comprising:
i) identifying an antibody having a basic affinity for the target antigen;
ii) genetically modifying a B-cell to encode the antibody identified in i);
iii) introducing the genetically modified B-cell in ii) to an animal expressing the target antigen and expressing a variant antigen that is structurally related to the target antigen;
iv) allowing the B-cells to undergo affinity maturation in the animal; and
v) isolating B-cells that have undergone affinity maturation in the animal;
whereby the B-cells isolated in v) express antibodies with improved specificity and/or affinity for the target antigen compared to the variant antigen when compared to antibodies expressed by the B-cells prior to affinity maturation.

27. The method of any of claims 1-26, wherein the target antigen is a conformationally-flexible antigen.

28. The method of claim 28, wherein the conformationally-flexible antigen is selected from the group consisting of: HIV envelope protein, circumsporozoite protein (CSP), merozoite surface protein 2 (MSP2) and GAD65.

29. A B-cell isolated according the method of any one of claims 1-28.

30. An isolated antibody expressed by the isolated B-cell according to claim 29.

31. A hybridoma that is derived from the isolated B-cell according to claim 29.

32. An isolated monoclonal antibody obtained from the hybridoma of claim 31.

33. A composition comprising the isolated antibody of claim 30 or claim 32.

34. The composition of claim 33, which is a therapeutic composition comprising a pharmaceutically acceptable carrier.

35. A transgenic animal comprising B-cells encoding an antibody for a target antigen, and wherein the animal has been genetically modified to express a variant antigen that is structurally related to the target antigen.

36. The transgenic animal of claim 35 which is a transgenic mouse or rat.

37. The transgenic animal of claim 35 or claim 36, wherein the B-cells have been genetically modified to encode the antibody for the target antigen.

38. The steps, features, integers, compositions and/or compounds disclosed herein or indicated in the specification of this application individually or collectively, and any and all combinations of two or more of said steps or features.


SEQUENCE LISTING (3 sequences)

SEQ ID NO: 1 - protein, 129 aa, Gallus gallus [sequence omitted]

SEQ ID NO: 2 - protein, 129 aa, artificial sequence
  mutant flexible HEL protein
KVFGRCELAAAMKRHGLDNYQGYSLGNWVCAAKFESNFNTQATNRNTDGSTDYGILQINSRWWCNDGRTPGSENLSNIPCSALLSSDITASVNSAKKIVSDGNGMNAWVAWRNRCKGTDVQAWIRGCRL

SEQ ID NO: 3 - protein, 129 aa, artificial sequence
  mutant rigid HEL protein
KVFGRCELAAAMKRHGLDNYQGYSLGNWVCAAKFESNFNTQATNRNTDGSTDYGILQINSRWWCNDGRTPGSENLCNIPCSALLSSDITASVNCAKKIVSDGNGMNAWVAWRNRCKGTDVQAWIRGCRL